SEQUENCE LISTING (18 sequences)

SEQ ID NO: 1 - protein, 57 aa, Homo sapiens
AYRPSETLCGGELVDTLQFVCGDRGFYFSRPASRVSRRSRGIVEECCFRSCDLALLE

SEQ ID NO: 2 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 3 - protein, 67 aa, Mus musculus
AYGPGETLCGGELVDTLQFVCSDRGFYFSRPSSRANRRSRGIVEECCFRSCDLALLETYCATPAKSE

SEQ ID NO: 4 - DNA, 20 bp, Artificial Sequence
  Synthetic oligonucleotide
cccattggtacctgaagttg

SEQ ID NO: 5 - DNA, 20 bp, Artificial Sequence
  Synthetic oligonucleotide
cgccttgtgatacgacttag

SEQ ID NO: 6 - protein, 70 aa, Mus musculus
GPETLCGAELVDALQFVCGPRGFYFNKLPGYGSSIRRAPQTGIVDECCFRSCDLRRLEMYCAPLKPTKAA

SEQ ID NO: 7 - DNA, 21 bp, Artificial Sequence
  Synthetic oligonucleotide
cccagcgagactctgtgcgga

SEQ ID NO: 8 - DNA, 21 bp, Artificial Sequence
  Synthetic oligonucleotide
ggaagtacggcctgagaggta

SEQ ID NO: 9 - DNA, 21 bp, Artificial Sequence
  Synthetic oligonucleotide
cgccgctagaggtgaaattct

SEQ ID NO: 10 - DNA, 21 bp, Artificial Sequence
  Synthetic oligonucleotide
cagacctccgactttcgttct

SEQ ID NO: 11 - DNA, 20 bp, Artificial Sequence
  Synthetic oligonucleotide
gaacatcatccctgcatcca

SEQ ID NO: 12 - DNA, 19 bp, Artificial Sequence
  Synthetic oligonucleotide
ccagtgagcttcccgttca

SEQ ID NO: 13 - DNA, 20 bp, Artificial Sequence
  Synthetic oligonucleotide
ccagcaggcggtgcatgaac

SEQ ID NO: 14 - DNA, 20 bp, Artificial Sequence
  Synthetic oligonucleotide
tcggagactaagcgcggcac

SEQ ID NO: 15 - DNA, 23 bp, Artificial Sequence
  Synthetic oligonucleotide
ggttccccacgttaggcttggat

SEQ ID NO: 16 - DNA, 21 bp, Artificial Sequence
  Synthetic oligonucleotide
ttgcggccctgggaatgagtg

SEQ ID NO: 17 - DNA, 20 bp, Artificial Sequence
  Synthetic oligonucleotide
gtccagctccatctgctcgc

SEQ ID NO: 18 - DNA, 19 bp, Artificial Sequence
  Synthetic oligonucleotide
cgtgccctctcgcagcttc

(19) United States
(12) Patent Application Publication
(10) Pub. No.: US 2012/0266263 A1
(43) Pub. Date: Oct. 18, 2012

(54) METHOD OF TREATING MEMORY DISORDERS AND ENHANCING MEMORY USING IGF-II COMPOUNDS

(75) Inventors: Cristina M. Alberini, New York, NY (US); Dillon Y. Chen, New York, NY (US)

(73) Assignee: MOUNT SINAI SCHOOL OF MEDICINE, New York, NY (US)

(21) Appl. No.: 13/502,313

(22) PCT Filed: Oct. 14, 2010
(86) PCT No.: PCT/US2010/052751

§ 371 Date: Jul. 3, 2012

Related U.S. Application Data
(60) Provisional application No. 61/251,625, filed on Oct. 14, 2009.

(51) Int. Cl. A61K 38/30; A61P 25/28; A01K 67/027; A61K 31/7088; A61P 25/16

(52) U.S. Cl. 800/12; 514/8.5; 514/44 R; 800/9

(57) ABSTRACT

The present invention provides compositions of insulin-like growth factor II (IGF-II) peptides or nucleic acids for the treatment of memory disorders and to enhance memory in subjects in need thereof.

### REFERENCE TO RELATED APPLICATIONS

The present application claims the benefit of priority to U.S. Provisional Patent Application No. 61/251,625 filed Oct. 14, 2009, the entire contents of which are incorporated herein.

### GOVERNMENT SPONSORED RESEARCH OR DEVELOPMENT

This invention was made in part in the course of research sponsored by an NIH Grants No. R01-MH065635 and R01-MH074736. The U.S. government has certain rights in the invention.

### FIELD OF THE INVENTION

The present invention provides compositions of insulin-like growth factor II (IGF-II) peptides or nucleic acids for the treatment of memory disorders and to enhance memory in subjects in need thereof.

### BACKGROUND OF THE INVENTION

The consolidation of newly learned information into a long-term memory depends upon a transient phase of mRNA and protein synthesis that occurs during a critical and limited time window initiated by training. This gene expression is, at least in part, evolutionarily conserved and involves members of the cAMP response element binding protein (CREB) and CCAAT enhancer binding protein (C/EBP) transcription factor families, which, in turns, control the transcription of downstream target genes (Alberini et al. (1994). Cell 76, pp. 1099-1114). The nature and identity of these target genes is still largely unknown. Studies in liver and other tissues show that C/EBP binding sites are present in tissue-specific promoters of IGF-II (Rodenburg et al. (1995). Mol. Endocrinol. 9, pp. 424-434; Shamblott et al. (1998). Mol. Mar. Biol. Biotechnol. 7, pp. 181-190), a growth factor that is expressed in the brain but still very poorly characterized.

IGF-II is a mitogenic polypeptide with structural homology to insulin and, together with insulin-like growth factor 1 (IGF-I), belongs to the IGF/IGFBP system (Russo et al. (2005). Endocr Rev. 26, pp. 916-943). IGF-II is poorly characterized: its expression is relatively high in the hippocampus, but is also found in the hypothalamus, striatum, cortex and cerebellum and found to decline with aging (Kitralo et al. (1993). Int J Dev Neurosci. 11, pp. 1-9). Most studies indicate that IGF-II and IGF-I play similar functions including promoting neuronal survival and protection against injury. IGF-II has been found to be involved in promoting neuronal survival, proliferation and maturation (DiCicco-Bloom and Black (1988). Proc. Natl. Acad. Sci., 85, pp. 4066-4070.), reduction of neuronal loss in adult brain following hypoxic-ischemic injury (Guan et al. (1993). J Cereb Blood Flow Metab. 13, pp. 609-616, Gluckman et al. (1992). J. Endocrinol. 134, pp. R1-3; Mackay et al. (2003). J Cereb Blood Flow Metab. 23, pp. 1160-1167), protection of oligodendrocytes and hippocampal septal neurons (McMorris and Dubois-Dalcq (1988). J. Neurosci Res. 21, pp. 199-209; Cheng and Mattson (1992). J. Neurosci. 12, pp. 1558-1566; Nicholas et al. (2002). J. Neuroimmunol. 124, pp. 36-44) neurite outgrowth (Ruiz et al. (1992). Arch Biochem Biophys. 296, pp. 231-238) and direct sprouting of spared afferent into a de-afferent hippocampus (Guthrie et al. (1995). J. Comp. Neurol. 352, pp. 147-160). While IGF-I has been found implicated in long-term synaptic plasticity and memory (Tropea et al. (2006). Nat. Neurosci. 9, pp. 660-668; Lupien et al. (2003). J. Neurosci. Res. 74, pp. 512-523), the function of IGF-II in these processes has not yet been characterized. Given that IGF-II is a putative C/EBP target gene, in this study, we employed rat IA to investigate whether the expression of IGF-II in the hippocampus is regulated by C/EBPb and involved in memory consolidation.

### SUMMARY OF THE INVENTION

In one embodiment, the present invention provides a method of enhancing memory strength in a subject in need thereof. The method comprises administering to the subject, a therapeutically effective amount of a compound selected from insulin-like growth factor II protein (IGF-II), a nucleic acid encoding IGF-II protein, an IGF-II peptide and a nucleic acid encoding an IGF-II peptide, or combination thereof.

In a further embodiment, the compound is administered by a route selected from the group consisting of intranasal, oral, topical, anal, ocular, optic, intramuscular, intraperitoneal, intravenous and intracerebral, or any combination thereof.

In another embodiment, a method of enhancing memory strength in a subject in need thereof is provided. The method comprises administering to the subject, a therapeutically effective amount of a the IGF-II protein comprising the sequence identified as SEQ ID NO: 1.

In yet another embodiment, a method of enhancing memory strength in a subject in need thereof is provided, wherein the subject has a memory impairment. The method comprises administering to the subject, a therapeutically effective amount of a compound selected from insulin-like growth factor II protein (IGF-II), a nucleic acid encoding IGF-II protein, an IGF-II peptide, a nucleic acid encoding an IGF-II peptide, or combination thereof.

In a further embodiment, the memory impairment is associated with neurodegenerative disease or aging, or the memory impairment is associated with head injury, spinal cord injury, seizure, stroke, epilepsy, ischemia, neuropsychiatric syndromes, CNS damage resulting from viral encephalitis. In some embodiments, the memory impairment is associated with Huntington's disease, Parkinson's disease, amyotrophic lateral sclerosis (ALS), neurodegeneration due to aging, or Alzheimer's disease

In yet another embodiment, a method for treating memory impairment in a subject in need thereof is provided. The method comprises administering to the subject, a therapeutically effective amount of a compound selected from insulin-like growth factor II protein (IGF-II), a nucleic acid encoding IGF-II protein, an IGF-II peptide, a nucleic acid encoding an IGF-II peptide, or a combination thereof.

In another embodiment, the present invention provides a method for treating memory loss in a subject in need thereof. The method comprises administering to the subject, a therapeutically effective amount of a human IGF-II protein comprising the sequence identified as SEQ ID NO: 1.

In yet another embodiment, a method for treating memory impairment in a subject in need thereof is provided, wherein the subject has a memory impairment associated with a neurodegenerative disease. The method comprises administering to the subject, a therapeutically effective amount of a compound selected from insulin-like growth factor II protein (IGF-II), a nucleic acid encoding IGF-II protein, an IGF-II peptide and a nucleic acid encoding an IGF-II peptide, or combination thereof.

In a further embodiment, the neurodegenerative disease is selected from Huntington's disease, Parkinson's disease, amyotrophic lateral sclerosis (ALS), neurodegeneration due to aging, and Alzheimer's disease. In another embodiment, the memory impairment is associated with head injury, spinal cord injury, seizure, stroke, epilepsy, ischemia, neuropsychiatric syndromes, CNS damage resulting from viral encephalitis, CNS damage resulting from meningitis, or CNS damage resulting from a tumor.

In one embodiment, a method of treating memory decay in a subject in need thereof is provided. The method comprises administering to the subject, a therapeutically effective amount of a compound selected from insulin-like growth factor II protein (IGF-II), a nucleic acid encoding IGF-II protein, an IGF-II peptide, a nucleic acid encoding an IGF-II peptide, or combination thereof. In a further embodiment, the subject has mild cognitive impairment.

In a further embodiment, the compound is administered by a route selected from the group consisting of intranasal, oral, topical, anal, ocular, optic, intramuscular, intraperitoneal, intravenous and intracerebral, or any combination thereof.

In one embodiment, the present invention provides a method of treating or preventing memory decay in a subject with mild cognitive impairment. The method comprises administering to the subject, a therapeutically effective amount of human IGF-II protein comprising the sequence identified as SEQ ID NO: 1.

In still another embodiment, a method for disrupting memory retention is provided. The method comprises administering to the subject an effective amount of a composition comprising an IGF-II antisense oligonucleotide.

In a further embodiment, a method for disrupting memory retention in an experimental animal is provided. The method comprises administering to the subject an effective amount of a composition comprising an IGF-II antisense oligonucleotide comprising the nucleic acid sequence identified as SEQ ID NO: 4.

In yet a further embodiment, the experimental animal is used to model Alzheimer's disease.

These and other embodiments are disclosed or are apparent from and encompassed by the following Detailed Description.

### BRIEF DESCRIPTION OF THE DRAWINGS

FIGS. 1A and 1B is a bar graph showing the quantification of a northern blot of rat hippocampal IGF-II mRNA at various time points after inhibitory avoidance training (IA).

FIG. 1C is a bar graph showing the results of a quantitative RT-PCR, where IGF-II mRNA levels were measured in rats subjected to IA training or the unpaired control protocol, as compared to 0 hr. controls.

FIG. 1D is a Western blot showing that the IGF-II antibody specifically recognizes IGF-II, as compared to IGF-I.

FIG. 1E is a bar graph showing the quantification of IGF-II expression (from a Western blot) in hippocampal extracts from rats that underwent IA training, as compared to control protocols. Data are expressed as mean percentage±SEM of the naïve control mean values (100%).

FIG. 2A is a bar graph showing the effect of IGF-II antisense administration to rat subjects after IA training, on IGF-II mRNA levels, compared to the effect of SC-ODN administration on IGF-II mRNA levels. Data are expressed as mean fold change±SEM of the ratio SC-ODN/IGF-II-ODN (**p<0.001).

FIG. 2B is a bar graph showing memory retention expressed as mean latency±SEM (seconds) in trained rats that received intrahippocampal single or double injections of either IGF-II-ODN or S-ODN/PBS.

FIG. 2C is a bar graph showing memory retention expressed as mean latency±SEM (seconds) in rats, after co-administration of either IGF-II-ODN or S-ODN with either recombinant IGF-II or recombinant IGF-I.

FIG. 3A is a bar graph showing memory retention expressed as mean latency±SEM (seconds) in IA trained rats that received a intrahippocampal double injection of IGF-II-ODN or S-ODN/PBS 24 and 32 hours after IA training, and tested 48 hours after training.

FIG. 3B is a bar graph showing memory retention expressed as mean latency±SEM (seconds) in IA trained rats that received intrahippocampal injection of IGF-II-ODN or S-ODN/PBS 96 and 104 hours after IA training, and tested 120 hours after training.

FIG. 4 shows results demonstrating that hippocampal post-training IGF-II administration enhances memory and prevents forgetting. Schedules are shown above figures. (A) Mean latency±SEM of trained rats given an hippocampal injection (↓) of vehicle, IGF-II or IGF-I and tested 24 h and 7-days later (two-way ANOVA F2,38=0.44, P>0.05 for interaction, F2,38=26.7, P<0.001 for treatment, F1,38=4.24, P<0.05 for test, Bonferroni post-hoc test **P<0.01, ***P<0.001). (B) Mean latency±SEM of trained rats given an hippocampal injection (↓) of vehicle or IGF-II (Student's t-test *P<0.05). (C) Mean % freezing of trained rats injected with vehicle or IGF-II (Student's t-test *P<0.05) (D) Mean latency±SEM of trained rats given bilateral amygdala injection (↓) of vehicle or IGF-II. (E) Mean latency±SEM of IA trained rats given an hippocampal injection (↓) of vehicle or insulin.

FIG. 5 shows results demonstrating post-retrieval IGF-II administration enhances memory and the effect is temporally limited. Schedules are shown above figures. (A) Mean latency±SEM of trained rats, tested 24 h post-training and, immediately afterward, injected (↓) with IGF-II, or IGF-I. Non-reactivated rats (NoR) were trained and injected (↓) without testing. Rats were tested at 48 h post-training (two-way ANOVA F_{1,26}=5.67, P<0.05 for interaction, F_{1,26}=9.82, P<0.01 for treatment, F_{1,26}=13.67, P<0.01 for test, Bonferroni post-hoc **P<0.01, *P<0.05). (B) Mean latency±SEM of trained rats, tested 14 d post-training and, immediately afterward, injected (↓) with vehicle or IGF-II and memory was tested 15 d after training.

FIG. 6 shows results demonstrating that IGF-II receptors are required for memory consolidation and IGF-II-mediated memory enhancement requires IGF-II receptors, de novo protein synthesis, and Arc. Schedules are shown above figures. (A) Mean latency±SEM of trained rats injected (↓) with vehicle, IGF-II, IGF-II/Anti-IGF2R, IGF-II/JB1, Anti-IGF2R, or JB1 (one-way ANOVA F5,40=3.82, P<0.01, Newman-Keuls post-hoc test *P<0.05 **P<0.01). (B) Mean latency±SEM of trained rats given double injections of IgG or anti-IGF-IIR antibody (Student's t-test **P<0.01). (C) Mean latency±SEM of trained and tested rats injected (↓) with vehicle, IGF-II or IGF-II+anisomycin (two-way ANOVA F2,34=5.25, P<0.05 for interaction, F2,34=4.68, P<0.05 for treatment, F1,34=13.7, P<0.001 for test, Bonferroni post-hoc **P<0.01,***P<0.001) (D) Mean latency±SEM of trained and tested rats injected (↓) with vehicle+SC-ODN, IGF-II+SC-ODN, or IGF-II+β-ODN (5 h: two-way ANOVA F2,26=2.8, P>0.05 for interaction, F2,26=5.93, P<0.01 for treatment, F1,26=11.9, P<0.01 for test, Bonferroni post-hoc **P<0.01; −1 h+5 h: two-way ANOVA F2,30=2.53, P>0.05 for interaction, F2,30=3.72, P<0.05 for treatment, F1,30=10.27, P<0.01 for test, Bonferroni post-hoc *P<0.05,**P<0.01) (E) Mean latency±SEM of trained and tested rats injected (↓) with vehicle+SC-ODN, vehicle+Arc-ODN, IGF-II+SC-ODN, or IGF-II+Arc-ODN (two-way ANOVA F1,18=7.8, P<0.05 for interaction, F1,18=17.3, P<0.001 for ODN-treatment, F1,18=12.3, P<0.01 for veh/IGF-II-treatment, Bonferroni post-hoc ***P<0.001).

FIG. 7 shows results demonstrating IGF-II-mediated memory enhancement requires GSK3 and is paralleled by an increase in synaptic expression of GluR1 and GSK3β activity and that IGF-II promotes LTP. (A) Western blot analysis of hippocampal pCREB and C/EBPβ rom naïve or trained rats injected (↓) with either vehicle or IGF-II and euthanized 20 h later (actin-normalized). Data are expressed as mean %±SEM of naïve-veh (one-way ANOVA, pCREB: F2,20=4.3, P<0.05, C/EBPβ: F2,19=5.7, P<0.05, Newman-Keuls post-hoc test, *P<0.05). (B) Western blot analysis of hippocampal GluR1 and GluR2 from naïve or trained rats injected (↓) with vehicle, IGF-II, IGF-II+Anti-IGF-IIR antibody (actin-normalized). Data are expressed as mean %±SEM of naïve-veh (one way ANOVA F3,19=4.24, *P<0.05, Newman-Keuls post hoc test *P<0.05). (C) Western blot analysis of hippocampal pGSK3β and GSK3β from the same extracts as in (B) (actin normalized). Data are expressed as mean %±SEM of naïve-veh (one-way ANOVA F3,19=4.93, *P<0.05, Newman-Keuls post hoc test *P<0.05,**P<0.01) (D) Mean latency±SEM of trained and tested rats injected (↓) with vehicle, IGF-II, SB216763 (SB) or IGF-II+SB (two-way ANOVA F3,56=4.44, P<0.01 for interaction, F3,56=5.07, P<0.01 for treatment, F1,56=9.12, P<0.01 for test, Bonferroni post-hoc **P<0.01,***P<0.001) (E). Time-courses of fEPSPs in area CA1 stratum radiatum are shown with sample traces obtained during the baseline period, 2 min after high frequency stimulation (HFS) and 100 min after HFS (gray traces: no HFS; black traces: HFS). Scale bars: 0.5 mV, 5 ms. Left panel: Weak HFS induced only transient potentiation that returned to baseline levels within 100 min (slope=109.4±9.7%, calculated as the average of the final 10 min of recording normalized to the full baseline period for each slice). Middle panel: In the presence of IGF-II, the same protocol induced stable LTP (135.2±6.6% of baseline, Student's t-test P<0.05). Right panel: In slices pretreated with antibodies against the IGFII receptor, IGF-II failed to facilitate the induction of stable LTP (112.05±4.7%).

### DETAILED DESCRIPTION OF THE INVENTION

The present inventors have found that administration of IGF-II enhances memory retention and treats memory impairment more effectively than administration of IGF-I. The difference between IGF-II and IGF-I in enhancing memory retention and treating memory impairment, for example, can be measured by hippocampal dependent learning assays that measure associate/contextual learning (e.g., Inhibitory Avoidance Assay, described in the Example section) and spatial learning (e.g., Morris Water Maze assay). Other memory tests have been described in the art (Squire (2004). Neurobiol Learn Mem. 82, pp. 171-177; Squire and Kandel, Memory: From Mind to Molecules, (2008). Roberts and Company Publishers; 2^{nd} Edition).

The superior result of IGF-II, as compared to IGF-I, in one embodiment, is measured in an inhibitory avoidance assay, and is a 2 fold increase in mean latency (measured in seconds) for a group of subjects administered IGF-II, as compared to group of subjects administered IGF-I. In another embodiment, the superior result of IGF-II, as compared to IGF-I, is measured in an inhibitory avoidance assay, and is a statistically significant difference in mean latency (measured in seconds) for a group of subjects administered IGF-II, as compared to group of subjects administered IGF-I.

Accordingly, the present invention provides IGF-II compounds, compositions, and methods for enhancing memory and/or treating memory impairment in subjects in need thereof. Additionally, methods for disrupting memory in an experimental animal are provided herein.

### DEFINITIONS

As used herein the term “IGF-II peptide” refers to a fragment of insulin-like growth factor II (IGF-II) protein which has a stronger effect on enhancing memory retention and/or treating memory loss than an equivalent amount of IGF-I protein.

“Treating” or “treatment” of a state, disorder or condition includes: preventing or delaying the appearance or slowing down the progression of clinical or sub-clinical symptoms of the state, disorder or condition developing in a mammal that may be afflicted with or predisposed to the state, disorder or condition but does not yet experience or display clinical or subclinical symptoms of the state, disorder or condition; or inhibiting the state, disorder or condition, e.g., arresting, reducing or delaying the development of the disease or a relapse thereof (in case of maintenance treatment) or at least one clinical or sub-clinical symptom thereof; or relieving the disease, e.g., causing regression of the state, disorder or condition or at least one of its clinical or sub-clinical symptoms. The benefit to a subject to be treated is either statistically significant or at least perceptible to the subject or to the person administering the treatment (e.g., a physician). For example, the benefit may be to increase memory retention, or increase memory strength, or slow down memory decay (e.g., as measured by the Inhibitory Avoidance assay or other tests disclosed herein).

“Patient” or “subject” refers to mammals and includes humans and veterinary animals.

“Experimental animal,” as used herein, refers to animals used in laboratory experiments, for example, chimpanzee, dog, cat, mouse, rat, rabbit, ferret, etc.

A “memory impairment,” as used herein, refers to a diminished level of mental registration, retention or recall of past experiences, knowledge, ideas, sensations, thoughts or impressions. Memory impairments may affect short and long-term information retention, facility with spatial relationships, memory (rehearsal) strategies, and verbal retrieval and production. The compositions and methods of the present invention can be used to ameliorate, treat and/or prevent memory impairments including, but not limited to, enhancing memory performance, improving or increasing the mental faculty by which to register, retain or recall past experiences, knowledge, ideas, sensations, thoughts, impressions, or a combination of the aforementioned.

For example, a memory impairment is a common feature of neurodegenerative diseases, such as Alzheimer's disease, Parkinson's disease or Huntington's disease. Memory disorders also occur with dementia, such as AIDS Dementia; Wernicke-Korsakoffs related dementia (alcohol induced dementia); age related dementia, multi-infarct dementia, a senile dementia caused by cerebrovascular deficiency, and the Lewy-body variant of Alzheimer's disease with or without association with Parkinson's disease. Creutzfeldt-Jakob disease, a spongiform encephalopathy caused by the prion protein, is a rare dementia with which memory disorder is associated. Loss of memory is also a common feature of brain-damaged patients. The term memory disorder is intended to cover mild cognitive impairment (MCI), which is a condition characterized by loss of memory, but no other symptoms of dementia, such as problems with language or personality or behavior changes. Patients with MCI are at high risk to develop other neurological diseases, such as Alzheimer's Disease.

Non-limiting examples of causes of brain damage which may result in a memory disorder include stroke, seizure, an anesthetic accident, ischemia, anoxia, hypoxia, cerebral edema, arteriosclerosis, hematoma, epilepsy, spinal cord cell loss, and peripheral neuropathy, head trauma, hypoglycemia, carbon monoxide poisoning, lithium intoxication, vitamin (B₁, thiamine and B₁₂) deficiency, excessive alcohol use, meningitis, a tumor and viral encephalitis.

“Memory decay,” as used herein, refers to a decrease or loss in memory, regardless of the cause. “Memory impairment” is a defective or aberrant memory. Memory decay may or may not be implicated in memory impairment.

“Memory loss,” as used herein, refers to a complete or partial loss of memory.

“Memory retention” is a measure of memory strength. Therefore, “enhancing memory strength” can be measured by a subject's ability to retain a particular memory.

“Memory reactivation,” as used herein, is synonymous with re-experiencing or retraining a particular memory. Assays for testing memory are provided herein.

“Long-term memory,” as used herein, refers to a memory that can last at least for a day, at least a year, at least a decade, or a lifetime.

A “therapeutically effective amount” means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The dosage of the therapeutic formulation will vary widely, depending upon the nature of the disease, the subject's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level.

In accordance with the present invention there may be employed conventional molecular biology, microbiology, protein expression and purification, antibody, and recombinant DNA techniques within the skill of the art. Such techniques are explained fully in the literature. See, e.g., Sambrook et al. (2001) Molecular Cloning: A Laboratory Manual. 3^{rd} ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.; Nucleic Acid Hybridization, Hames & Higgins eds. (1985); Transcription And Translation, Hames & Higgins, eds. (1984); Animal Cell Culture Freshney, ed. (1986); Immobilized Cells And Enzymes, IRL Press (1986); Perbal, A Practical Guide To Molecular Cloning (1984); and Harlow and Lane. Antibodies: A Laboratory Manual (Cold Spring Harbor Laboratory Press: 1988).

### Compounds of the Invention

The IGF-II proteins and peptides of the present invention can be produced by recombinant DNA methods, well known to one of ordinary skill in the art. For example, an IGF-II DNA sequence, can be used in an expression construct, to express the full length protein, or a fragment thereof.

In one embodiment, the IGF-II protein of the present invention has the following sequence:

| (SEQ ID NO: 1) | |
|---|---|
| 25 | ayrpse tlcggelvdt lqfvcgdrgf yfsrpasrvs |
| 61 | rrsrgiveec cfrscdlall e. |

SEQ ID NO: 1 is a fragment of the human IGF-II sequence, corresponding to Ala₂₅-Glu₉₁. The IGF-II protein can also be full length IGF-II, as provided by Genbank accession No. P01344, and SEQ ID NO: 2, below:

(SEQ ID NO: 2)
mgipmgksmlvlltflafascciaayrpsetlcggelvdtlqfvcgdrgfyfsrpasrvsrr
srgiveeccfrscdlalletycatpakserdvstpptvlpdnfprypvgkffqydtwkqstq
rlrrglpallrarrghvlakeleafreakrhrplialptqdpahggappemasnrk.

Other IGF-II peptide fragments can be generated from SEQ ID NO: 2, and are within the scope of the invention.

In another embodiment, the IGF-II protein of the present invention has the following sequence.

(SEQ ID NO: 3)
AYGPGETLCGGELVDTLQFVCSDRGFYFSRPSSRANRRSRGIVEECCFRSCDLALLETYCAT
PAKSE

In some embodiments, fragments of the IGF-II protein can be used in the methods of the present invention, as long as such fragments have a stronger effect on enhancing memory retention and/or treating memory loss than an equivalent amount of IGF-I protein.

Additionally, other IGF-II proteins and peptides are within the scope of the invention. For example, the following IGF-II proteins, and peptide fragments thereof, can be used in the methods, compounds and compositions of the invention. In one embodiment, the IGF-II sequence corresponds to the sequence provided by Genbank Accession Nos.: P09535 (mouse), P01346 (rat), P01344 (human), P23695 (pig), P07456 (cow), P33717 (chicken), P51459 (horse), P10764 (sheep), AAI56000 (Xenopus), AAI70810 (Xenopus), AAI70812 (Xenopus), or a fragment thereof IGF-II sequences are known in the art, and depending on the subject to be treated, one of ordinary skill in the art will know which source (e.g., human, mouse, rat, etc.) of IGF-II to use.

An expression construct is a nucleic acid sequence comprising a target nucleic acid sequence (e.g., IGF-II) whose expression is desired, operatively associated with expression control sequence elements which provide for the proper transcription and translation of the target nucleic acid sequence(s) within the chosen host cells. Such sequence elements may include a promoter and a polyadenylation signal. The expression construct may further comprise vector sequences. Vector sequences are any of several nucleic acid sequences established in the art which have utility in the recombinant DNA technologies of the invention to facilitate the cloning and propagation of the expression constructs including (but not limited to) plasmids, cosmids, phage vectors, viral vectors, and yeast artificial chromosomes.

Expression constructs of the present invention may comprise vector sequences that facilitate the cloning and propagation of the expression constructs which include IGF-II nucleic acid or fragment thereof. A large number of vectors, including plasmid and fungal vectors, have been described for replication and/or expression in a variety of eukaryotic and prokaryotic host cells. Standard vectors useful in the current invention are well known in the art and include (but are not limited to) plasmids, cosmids, phage vectors, viral vectors, and yeast artificial chromosomes. The vector sequences may contain a replication origin for propagation in E. coli; the SV40 origin of replication; an ampicillin, neomycin, or puromycin resistance gene for selection in host cells; and/or genes (e.g., dihydrofolate reductase gene) that amplify the dominant selectable marker plus the gene of interest.

The IGF-II DNA sequence is expressed in or by a cell to form an IGF-II expression product, such as a protein or peptide. The expression product itself (the resulting protein or peptide), may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

Once the IGF-II DNA sequence is inserted into a vector, or formulated into another expression construct, it is transformed into a host cell for expression as the respective protein or peptide. A host cell that receives and expresses introduced DNA or RNA has been transformed and is a transformant or a clone. The DNA or RNA introduced to a host cell can come from any source, including cells of the same genus or species as the host cell, or cells of a different genus or species.

### Antisense Compounds

The present invention also provides IGF-II antisense oligonucleotide (IGF-II-ODN) to model memory loss in an experimental animal. In one embodiment, the antisense oligonucleotide has the sequence:

(SEQ ID NO: 4)
5′-CCCATTGGTACCTGAAGTTG-3′

However, other antisense oligonucleotides, specific for IGF-II can be used to disrupt memory in an experiment animal.

### Compositions of the Invention

The compositions and compounds of the present invention include peptides, proteins and nucleic acid molecules, and may be formulated for administration in any convenient way for use in human or veterinary medicine. The invention therefore includes within its scope pharmaceutical compositions comprising a compound of the invention adapted for use in human or veterinary medicine.

The compounds and compositions can be administered orally, intranasally, topically or anally. Additionally, the compounds and compositions presented herein can be administered via a parenteral route. For example, the compounds can be administered by a route selected from intramuscular, intraperitoneal, intravenous and intracerebral, or any combination thereof.

In a further embodiment, the compounds and compositions of the present invention are administered by a route selected from the group consisting of intranasal, oral, topical, anal, ocular, optic, intramuscular, intraperitoneal, intravenous and intracerebral, or any combination thereof.

The compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro, 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used.

The compounds used in the invention may be milled using known milling procedures such as wet milling to obtain a particle size appropriate for tablet formation and for other formulation types. Finely divided (nanoparticulate) preparations of the compounds may be prepared by processes known in the art, for example see International Patent Application No. WO 02/00196 (SmithKline Beecham).

The compounds and pharmaceutical compositions of the present invention can be administered orally (e.g., as a tablet, sachet, capsule, pastille, pill, boluse, powder, paste, granules, bullets or premix preparation, ovule, elixir, solution, suspension, dispersion, gel, syrup or as an ingestible solution).

Additionally, the compounds presented herein can be formulated for parenteral administration (e.g., intramuscular, intraperitoneal, intravenous, intracerebral). Compounds may be present as a dry powder for constitution with water, PBS, or other suitable vehicle before use, optionally with flavoring and coloring agents. Solid and liquid compositions may be prepared according to methods well-known in the art. Such compositions may also contain one or more pharmaceutically acceptable carriers and excipients which may be in solid or liquid form.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable dyes useful for the compositions of the present invention include, but are not limited to, synthetic and natural dyes such as titanium dioxide, beta-carotene and extracts of grapefruit peel.

Examples of pharmaceutically acceptable coatings useful for the oral compositions of the present invention, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable buffers useful for the compositions of the present invention include, but are not limited to, citric acid, sodium citrate, sodium bicarbonate, dibasic sodium phosphate, magnesium oxide, calcium carbonate and magnesium hydroxide.

Examples of pharmaceutically acceptable surfactants useful for the oral and parenteral compositions of the present invention include, but are not limited to, sodium lauryl sulfate and polysorbates.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Representative examples of pharmaceutically acceptable stabilizers and antioxidants for use in the present invention include, but are not limited to, ethylenediaminetetriacetic acid (EDTA), thiourea, tocopherol and butyl hydroxyanisole.

The pharmaceutical compositions of the invention may contain from 0.01 to 99% weight per volume of the active material (i.e., the IGF-II compound).

### Methods of the Invention

### Memory Enhancement

In one embodiment, the present invention provides IGF-II compounds, IGF-II compositions, and methods of using such compounds and compositions for ameliorating memory diseases or memory impairment. Thus, the compositions and methods of the present invention can be used to prevent, delay onset, or treat memory impairment. The present invention can increase mental registration, retention or recall of past experiences, knowledge, ideas, sensations, thoughts or impressions. In a preferred embodiment, the present invention increases short and/or long-term information retention, facility with spatial relationships, memory (rehearsal) strategies, and verbal retrieval and production.

The compounds, compositions and methods of the present invention can improve hippocampal-dependent learning. In one embodiment, the compositions and methods of the present invention can improve associative learning. In another embodiment, the compositions and methods of the present invention can improve spatial memory. As shown in the examples, the effects of centrally administered IGF-II on contextual memory, which is hippocampal dependent, was investigated using the inhibitory avoidance (IA) paradigm in rats. Surprisingly, the inventors discovered that the IGF-II protein significantly increased memory retention, compared to the administration of IGF-I protein.

In a further embodiment, the present invention provides of a method for enhancing memory strength in a subject in need thereof, comprising administering to the subject, a therapeutically effective amount of a compound selected from IGF-II, a nucleic acid encoding IGF-II protein, an IGF-II peptide and a nucleic acid encoding an IGF-II peptide, or combination thereof.

In yet a further embodiment, the present invention provides of a method for enhancing memory strength in a subject in need thereof, comprising administering to the subject, a therapeutically effective amount of insulin-like growth factor II protein (IGF-II) comprising the sequence identified by SEQ ID NO: 1. In another embodiment, the IGF-II protein is selected from the sequence provided by Genbank Accession No. P01346, P01344, P23695, P07456, P33717, P51459, P10764, AAI56000, AAI70810 or AAI70812, or a fragment thereof.

### Therapeutic Uses

In one embodiment, the IGF-II compounds and compositions provided herein are used to treat memory impairment in a subject in need thereof. In a further embodiment, the memory impairment is associated with neurodegenerative disease or aging, or the memory impairment is associated with head injury, spinal cord injury, seizure, stroke, epilepsy, ischemia, neuropsychiatric syndromes, CNS damage resulting from viral encephalitis. In some embodiments, the memory impairment is associated with Huntington's disease, Parkinson's disease, amyotrophic lateral sclerosis (ALS), neurodegeneration due to aging, or Alzheimer's disease.

In a preferred embodiment, the subject suffers from Alzheimer's disease. Alzheimer's disease (AD) is a degenerative brain disease, the incidence of which rapidly increases with advancing age. Recently modern imaging techniques have revealed how the medial temporal lobe area, which contains the hippocampus (a vital structure for learning and memory generally in humans and for certain types of spatial learning in animals) progressively shrinks as Alzheimer's disease runs its course. The principle symptoms of Alzheimer's disease are steadily progressive loss of cognitive faculties such as memory (particularly recent episodic memories), problems with language and speech such as difficulty in finding the right words, and attention. Multi-infarct dementia, the most common other form of dementia, often presents a similar clinical picture but as it is due to a series of small strokes its progression is more stepwise. Accordingly, in one embodiment, IGF-II compounds and compositions of the present invention are used to delay onset, amerliorate the symptoms, treat the symptoms of Alzheimer's disease, or treat Alzheimer's disease. In another embodiment, the IGF-II protein is selected from the sequence provided by Genbank Accession No. P01346, P01344, P23695, P07456, P33717, P51459, P10764, AAI56000, AAI70810 or AAI70812, or a fragment thereof.

In one embodiment, the methods and compositions of the present invention can be used to slow or stop the conversion from MCI to Alzheimer's disease.

### Disrupting Memory in Experimental Animals

In one embodiment, the present invention provides compounds, compositions and methods for disrupting memory in an experimental animal. The method can comprise, administering to an experimental animal a composition comprising an IGF-II antisense oligonucleotide. In one specific embodiment, the method can comprise, administering to an experimental rat a composition comprising a rat IGF-II antisense oligonucleotide comprising the nucleic acid sequence identified as SEQ ID NO: 4. In still a further embodiment, the experimental animal is used as a model of Alzheimer's disease (e.g., to test therapies for Alzheimer's disease).

### Delivery Methods

Various delivery systems are known and are used to administer a therapeutic of the invention, e.g., encapsulation in liposomes, microparticles, microcapsules, expression by recombinant cells, receptor-mediated endocytosis (see, e.g., Wu & Wu, J. Biol. Chem. 265:4429-4432, 1987), construction of a therapeutic nucleic acid as part of a viral or other vector, etc.

In an embodiment where the therapeutic is a nucleic acid encoding an IGF-II protein or peptide, the nucleic acid can be administered in vivo to promote expression of IGF-II protein or peptide, by constructing it as part of an appropriate nucleic acid expression vector and administering it so that it becomes intracellular, e.g., by use of a viral vector (see U.S. Pat. No. 4,980,286), or by direct injection, or by use of microparticle bombardment (e.g., a gene gun; Biolistic, Dupont) or coating with lipids or cell-surface receptors or transfecting agents, or by administering it in linkage to a homeobox-like peptide that is known to enter the nucleus (see, e.g., Joliot et al. (1991). Proc. Natl. Acad. Sci., U.S.A. 88, pp. 1864-1868). Alternatively, a nucleic acid therapeutic can be introduced intracellularly and incorporated within host cell DNA for expression by homologous recombination.

Other methods for improving the delivery and administration of the pharmacological agent of the present invention include means for improving the ability of the pharmacological agent to cross membranes, and in particular, to cross the blood-brain barrier. One skilled in the art can readily assay the ability of the IGF-II compound to cross the blood-brain barrier in vivo, for example using a model of the blood-brain barrier based on a brain microvessel endothelial cell culture system (see, e.g., Bowman et al., (1983). Ann. Neurol. 14, pp. 396-402; Takahura et al. (1992). Adv. Pharmacol. 22, 137-165). In one embodiment, the pharmacological agent can be modified to improve its ability to cross the blood-brain barrier, and in an alternative embodiment, the pharmacological agent can be co-administered with an additional agent, such as for example, an anti-fungal compound, that improves the ability of the pharmacological agent to cross the blood-brain barrier (see Pardridge (2002). W. M. Neuron 36, pp. 555-558).

Methods of introduction include, but are not limited to, intranasal, oral, topical, anal, ocular, optic, intramuscular, intraperitoneal, intravenous and intracerebral routes, or any combination thereof.

The compounds and compositions of the invention can be administered together with other biologically active agents (e.g., other therapeutics which improve memory and/or treat memory impairment).

### EXAMPLES

The present invention is further illustrated by reference to the following Examples. However, it should be noted that these Examples, like the embodiments described above, are illustrative and are not to be construed as restricting the enabled scope of the invention in any way.

### Materials and Methods

### Animals

Long Evans adult male rats (Harlan, Indianapolis, Ind.) weighing between 200 and 250 grams at the beginning of the experiments were used. Rats were housed individually on a 12 hour light-dark cycle with ad libitum access to food and water. All experiments were conducted during the light cycle between 9 AM and 6 PM. All protocols complied with the National Institute of Health Guide for the Care and Use of Laboratory Animals.

### Inhibitory Avoidance (IA)

IA was carried out as previously described (Garcia-Osta et al. (2006). J. Neurosci. 26, pp. 7919-7932). The IA chamber consisted of a rectangular-shaped Perspex box, divided into two compartments—(1) a safe compartment and (2) a shock compartment. The safe compartment was white and illuminated, while the shock compartment was black and dark. Foot shocks were delivered to the grid floor of the shock chamber via a constant current scrambler circuit. The IA chamber was located in a sound-attenuated, non-illuminated room. During training sessions, each rat was placed in the safe compartment with its head facing away from the door. After 10 seconds, the door separating the compartments was automatically opened, allowing the rat access to the shock compartment. The door closed 1 second after the rat entered the shock compartment, and a brief foot shock (0.6 mA for 2 seconds) was administered. The time (i.e., latency) to enter the shock compartment was taken in seconds as acquisition.

The rat was then returned to its home cage and tested for memory retention 24 hours later. Retention tests were performed by placing the rat back into the safe compartment and measuring its latency to enter the shock compartment. Foot shocks were not administered on the retention tests, and testing was terminated at 540 seconds or 900 seconds as indicated.

Where specified, control consisted of: either rats exposed to the training apparatus without foot shock, or rats exposed to the training apparatus and subsequently to the foot shock 1 hour later (unpaired, unp).

In the reactivation (reconsolidation experiments), rats were trained as described above and at the indicated time points they were tested. This test reactivated the memory. Immediately after the subjects were injected with the indicated compounds, each was subsequently tested again for retention. Statistical analyses were performed using a one-way analysis of variance (ANOVA) followed by Newman-Keuls post hoc tests.

### Contextual and Auditory Fear Conditioning

Fear conditioning was carried out as previously described. Muravieva et al., (2010) Learn Mem 17 pp. 306-313. Rats were conditioned in contextual fear conditioning chamber (CFC), which consisted of a rectangular Plexiglass box (30.5 cm×24.1 cm×21.0 cm) with a metal grid floor (Model ENV-008 Med Associates). All rats were pre-exposed to this chamber for 5 min. On the next day, rats were placed in CFC chamber for 120 sec and then presented with 30 sec of the auditory cue consisting of a 5 kHz 75 dB tone that co-terminated with a 0.6 mA 2-sec footshock. One hundred twenty sec after the first footshock, another 30 sec auditory cue was presented that also co-terminated with another 0.6 mA 2-sec footshock. Rats were returned to their home cage 120 sec after the second footshock. Freezing levels during the first 148 sec (before the presentation of the first footshock) was recorded, scored and reported as baseline freezing. Freezing was defined as lack of movement except for breathing. Twenty-four hour later, rats were placed back in the CFC chamber and their freezing levels recorded for 5 min and scored. Twenty-four hours after CFC test, rats were placed in a different context (the illuminated IA box) for 120 sec before being presented with three 30 sec auditory cues. The three 30 sec auditory cues were separated by 120 sec. Freezing levels during the cue presentations was recorded and scored by an experimenter who was blind to the treatment conditions.

### Oligodeoxynucleotide and Recombinant Protein Hippocampal and Amygdala Injections

Hippocampal and amygdala injections were given as previously described (Garcia-Osta et al. (2006). J. Neurosci. 26, pp. 7919-7932; Taubenfeld et al. (2001). J. Neurosci. 21, pp. 84-91; Tronel et al. (2007). Biol Psychiatry 62 pp. 33-39). Rats were anesthetized with sodium pentobarbital (55 mg/kg, i.p.) or ketamine (65 mg/kg, i.p.) and xylazine (7.5 mg/kg, i.p.), and stainless-steel guide cannulae (22-gauge) were stereotactically implanted to bilaterally target the hippocampus (4.0 mm posterior to the bregma; 2.0 mm lateral from midline; and 2.0 mm ventral). For amygdala injections, 26-gauge guide cannuale were implanted to bilaterally target the basolateral amygdala (2.8 mm posterior to bregma; 5.3 mm lateral from midline; and 6.25 mm ventral). The rats were returned to their home cages and allowed to recover from surgery for 7 days.

At the indicated time points before or after training or retrieval, rats received bilateral injections of compounds as specified. All injections are indicated by arrow in the experimental schedule. All hippocampal injections were carried out in 1 μl per side, whereas all amygdala injections were done in 0.5 μl per side. Hippocampal injections used a 28-gauge needle and amygdala injections used 33-gauge needles that extended 1.5 mm beyond the tip of the guide cannula and connected via polyethylene tubing to a Hamilton syringe. The infusions were delivered at a rate of 0.33 μl/min using an infusion pump. The injection needle was left in place for two min after the injection to allow complete dispersion of the solution.

To verify proper placement of cannula implants, at the end of the behavioral experiments, rats were anesthetized and perfused with 4% paraformaldehyde in PBS. Their brains were postfixed overnight in the same fixative with 30% sucrose. Forty-micrometer coronal sections were cut through the hippocampus, stained with cresyl violet, and examined under a light microscope. Rats with incorrect cannula placement were discarded from the study.

Antisense ODNs and relative scrambled sequences (SC-ODNs) were injected at 2 nmol/μl in all antisense experiments.

Sequences:
C/EBPβ antisense
(SEQ ID NO: 13)
(β-ODN: 5′-CCAGCAGGCGGTGCATGAAC-3)
C/EBPβ scrambled
(SEQ ID NO: 14)
(SC-ODN: 5′-TCGGAGACTAAGCGCGGCAC-3′)
rat IGF-II antisense
(SEQ ID NO: 4):
(IGF-II-ODN: 5′-CCCATTGGTACCTGAAGTTG-3′)
IGF-II scrambled
(SEQ ID NO: 5)
(SC-ODN: 5′- CGCCTTGTGATACGACTTAG-3′);
Arc antisense
(SEQ ID NO: 17)
(Arc-ODN: 5′-GTCCAGCTCCATCTGCTCGC-3′)
Arc scrambled
(SEQ ID NO: 18)
(SC-ODN: 5′-CGTGCCCTCTCGCAGCTTC-3′)

Vehicle was Phosphate-Buffered Saline (PBS, pH 7.4).

The antisense for C/EBPβ has been previously shown to knock-down C/EBPβ in the hippocampus. The antisense for IGF-II mRNA was specific for the sequence that includes the translational start site and was previously used successfully to knockdown IGF-II in other tissues (Elizalde et al. (1998). J Steroid Biochem Mol. Biol. 67, pp. 305-317). The antisense for Arc has been previously shown to block Arc protein expression and long-term memory consolidation when injected into the hippocampus (Guzowski et al. (2000) J. Neurosci. 20, pp. 3993-4001). The respective SC-ODNs, which served as control, contained the same base composition but in a random order and show no homology to sequences in the GenBank database. All ODNs were phosphorothioated on the three terminal bases of both 5′ and 3′ ends to produce increased stability. Both ODNs were RPC (Reverse-Phased-Cartridge)-purified and obtained from Gene Link (Hawthorne, N.Y.).

Recombinant IGF-I and IGF-II were purchased from R&D Systems (Minneapolis, Minn.) and were dissolved in 0.1% bovine serum albumin (BSA) in 1×PBS.

The following IGF-I sequence was used (Gly33 to Ala102, Genbank Accession No. Q8CARO.

(SEQ ID NO: 6)
GPETLCGAELVDALQFVCGPRGFYFNKLPGYGSSIRRAPQTGIVDECCFR
SCDLRRLEMYCAPLKPTKAA

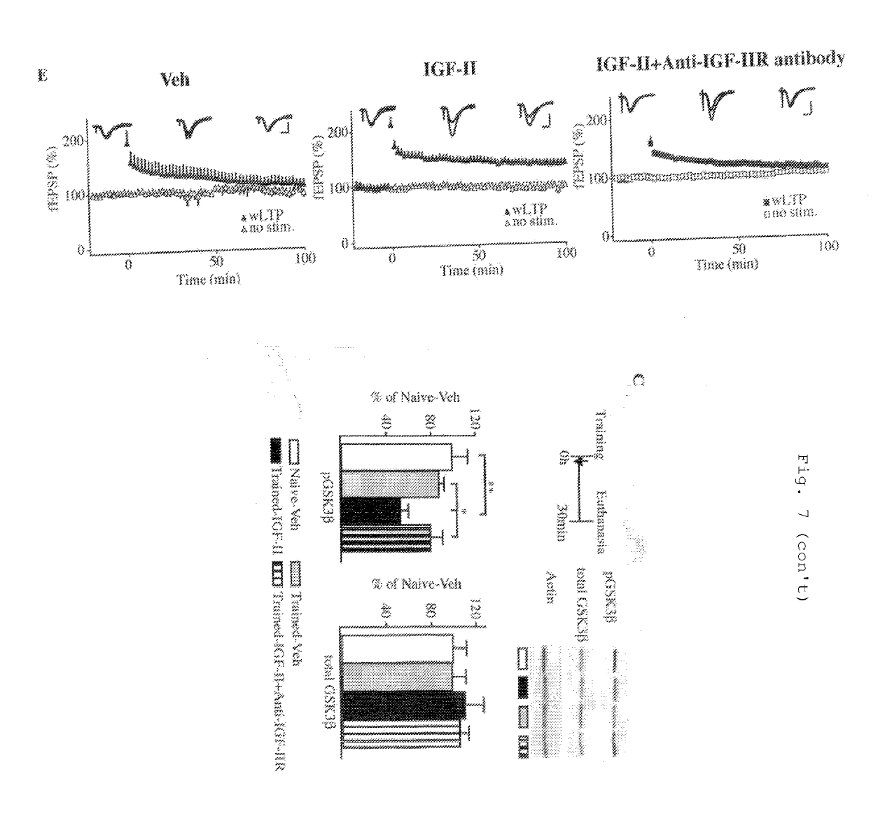

All experiments with recombinant IGF-II or IGF-I were carried out with 250 ng/injection, except for the dose-response curve (250, 25 or 2.5 ng) except for those in FIG. 6A, where 25 ng was used.

The IGF-I receptor (IGF-1R) antagonist JB-1 (Bachem Biosciences, Torrance, Calif.) was dissolved in PBS. JB-1 was injected at 20 ng/μl, a concentration that has been used successfully to block IGF-1 activity in various tissues including the brain. Anti-IGF-II receptor antibody (anti-IGF2R, R&D Systems, Minneapolis, Minn.) was dissolved in 1×PBS and injected at 5 ng/μl. This concentration blocked 95% of IGF-II receptor in an in-vitro binding assay (R&D).

Anisomycin (Sigma Aldrich, St. Louis, Mo.) was dissolved in 0.9% saline pH 7.4. and injected at 125 μg/μl of anisomycin. This dose blocks more than 80% of protein synthesis in the dorsal hippocampus for up to 6 h.

GSK3 inhibitor SB216763 was purchased from Sigma and was dissolved in 1% DMSO in PBS and injected at 1 ng/μl. This dose blocks has been shown to block GSK3β activity (as measured by its dephosphorylation levels) in the brain.

### Synaptoneurosomal Preparation and Western Blot Analysis

Synaptoneurosomal preparation was adapted from Elkobi et al. (2008) Nat. Neurosci 11, pp. 1149-1151. Briefly, dorsal hippocampi were rapidly dissected in ice-cold cortical dissection buffer followed by homogenization in buffer containing 10 mM HEPES, 2 mM EDTA, 2 mM EGTA, 0.5 mM DTT, phosphatase inhibitor cocktail and protease inhibitor cocktail. Glass-teflon homogenizer was used and homogenates were filtered through 100 μm and 5 μm filters sequentially. Synaptoneurosomes were obtained by centrifuging the filtrate at 1000 g for 10 min. Synaptoneurosomal fraction was enriched with PSD-95 and N-methyl-D-aspartic acid (NMDA) receptor subunit NR-1.

Western blot analysis was carried as previously reported (Taubenfeld et al. (2001). J. Neurosci. 21, pp. 84-91; Garcia-Osta et al. (2006). J. Neurosci. 26, pp. 7919-7932). Hippocampal total extracts from rat were obtained by polytron homogenization in cold lysis buffer with protease inhibitors (0.2 M NaCl, 0.1 M HEPES, 10% glycerol, 2 mM NaF, 2 mM Na₄P₂O₇, aprotonin 4 U/ml, DTT 2 mM, Leupeptin, 10 μg/ml, EGTA 1 mM, microcystin, 1 μM, benzamidine, 1 mM). Protein concentrations were determined using the BioRad protein assay (BioRad Laboratories, Hercules, Calif.). Equal amounts of total protein (10-20 μg/lane) were resolved on denaturing 10% SDS-PAGE gels and transferred to Hybond-P membranes (Millipore, Billenca, Mass.) by electroblotting. Membranes were dried for at least 30 min at room temperature, immerged in methanol for 5 min and then washed with 3 changes of water. The membrane was then blocked in 3% milk/PBS or according to manufacturers' instruction for 1 h at room temperature, then incubated with either anti-IGF-II (1/500, Millipore, Billerica, Mass.), or anti-actin (1/5000, Santa Cruz Biotechnology, Santa Cruz, Calif.) antisera in PBS overnight at 4° C. Anti-phospho-CREB (1/1000), anti-GluR1 (1/2000), anti-GluR2 (1/2000), anti-PSD95 (1/5000) and anti-NR1 (1/1000) antibodies were purchased from Millipore. Anti-C/EBPβ antibody was purchased from Santa Cruz Biotechnology (1/1000, Santa Cruz, Calif.). pGSK3β and GSK3β antibodies were purchased from Cell Signaling (1/1000, Danvers, Mass.) pGSK3β was normalized to actin and GSK3β. The colloidal gold total protein stain was purchased from BioRad. The membranes were washed, treated with a secondary HRP-labeled donkey anti-rabbit antibody (1/4000, GE Healthcare, Waukesha, Wis.) for 1 h, washed again and incubated with HRP-streptavidin complex and ECL detection reagents (GE healthcare, Waukesha, Wis.). Membranes were exposed to Denville Scientific HyBlotCL (Denville Scientific, Metuchen, N.J.), and quantitative densitometric analysis was obtained using NIH Image J.

Real Time Quantitative RT-PCR (qRT-PCR)

Hippocampal total RNA was extracted with TRizol® Reagent (Invitrogen, Carlsbad, Calif.) and reverse transcribed using SuperScript® II RNAse H minus RT (Invitrogen). Real time PCR was performed with an ABI Prism 7900HT (Applied Biosystems, Foster City Calif.). 500 pg of the first-strand cDNA was subjected to PCR amplification using Quantitect SYBR Green PCR kit (Qiagen, Valencia, Calif.). Specific primers were designed based on GenBank accession numbers). GAPDH or 18S rRNA were used as internal controls.

Forty cycles of PCR amplification were performed as follows: denature at 95° C. for 30 seconds, anneal at 55° C. for 30 seconds and extend for 30 seconds at 72° C. Three PCR assays were performed in triplicate for each cDNA sample.

Rat IGF-II (Genback accession no: NM_031511)
(SEQ ID NO: 7)

| | |
|---|---|
| Forward primer: | CCCAGCGAGACTCTGTGCGGA |

(SEQ ID NO: 8)

| | |
|---|---|
| Reverse primer: | GGAAGTACGGCCTGAGAGGTA |

18S rRNA (Genback accession no: M11188)
(SEQ ID NO: 9)

| | |
|---|---|
| Forward primer: | CGCCGCTAGAGGTGAAATTCT |

(SEQ ID NO: 10)

| | |
|---|---|
| Reverse primer: | CAGACCTCCGACTTTCGTTCT |

GAPDH (Genback accession no: M17701)
(SEQ ID NO: 11)

| | |
|---|---|
| Forward primer: | GAACATCATCCCTGCATCCA |

(SEQ ID NO: 12)

| | |
|---|---|
| Reverse primer: | CCAGTGAGCTTCCCGTTCA |

Data were analyzed with Sequence Detector System version 2.0 software (Applied Biosystems, Foster City Calif.). The cycle threshold method (C_{T}, see Applied Biosystems User Bulletin Number 2, P/N 4303859) was chosen to determine the relative quantification of gene expression in trained and control rats.

### Chromatin Immunoprecipitation (ChIP)

ChIP was performed as described in Tsankova et al. (2004) 24, J. Neurosci. pp. 5603-5610. The rat hippocampi were dissected and minced into ˜1 mm pieces, and immediately cross-linked in 1% formaldehyde for 17 min at room temperature rotating. The cross-linking reaction was stopped by adding glycine to a final concentration of 0.125 M and incubated for 7 min. The tissue was washed five times in cold PBS containing protease inhibitor (Roche Applied Sciences, Indianapolis, Ind.) and then frozen on dry ice. The chromatin was solubilized and extracted by adding 500 μl of lysis buffer (1% SDS, 50 mM Tris-HCl pH8.1, mM EDTA), followed by sonication. The homogenate was diluted in 1.1 ml ChIP dilution buffer (1.1% Triton X-100, 167 mM NaCl, 16.7 mM Tris-HCL pH8.1, 1.2 mM EDTA, 0.01% SDS). The homogenate was used for C/EBPβ ChIP. 30 μl of Magnetic Protein A beads (EZ-Magna ChIP A kit, Millipore) and 5 μg of C/EBPβ antibody was added to the homogenate. The mixture was incubated rotating overnight in 4° C. The wash, elution, and reverse cross-link to free DNA were all performed according to the manufacturer's protocol (EZ-Magna ChIP A kit).

Specific primers were designed to amplify the proximal promoter region of approximately 150 bp 5′ of exon one (GenBank: X17012.1), which contains a putative C/EBP binding site. Putative C/EBP binding site was predicted using an on-line program AliBaba 2.1. Similar C/EBP binding sites have been identified in other species. Primer sequences used: forward GGTTCCCCACGTTAGGCTTGGAT (SEQ ID NO: 15); reverse TTGCGGCCCTGGGAATGAGTG (SEQ ID NO: 16). A standard thirty-five cycle PCR was performed as followed: denature at 95° C. for 30 sec, anneal at 58° C. for 30 sec and extend for 30 sec at 72° C. The PCR reaction was resolved on a 2% agarose gel and sequenced. Sequencing confirmed the identity of the fragment. DNA sequencing was performed by W.M. Keck Facility at Yale University, New Haven, Conn.

### Northern Blot Analysis

Northern blot analyses were performed as previously described (Taubenfeld et al. (2001). Nat. Neuroscience 4, pp. 769-781). The rat IGF-II probe consisted of a 224 bp fragment that corresponds to nucleotides 1145-1368 of the IGF-II sequence in GenBank accession number NM-031511. The same membrane was stripped and re-hybridized with a full-length rat cyclophilin probe which was used as a loading control. Probes were labeled with random oligonucleotides primers (Prime-It II kit, Stratagene) and [α-³²P]dCTP (Amersham). Quantitative densitometry analysis was performed using NIH image. Data were expressed as mean percentage±SEM of the 0 h (100%) control mean values. Statistical analyses were carried out using Student t test or a one-way ANOVA followed by Newman-Keuls post hoc test.

### Electrophysiology Methods

Brains were removed from isoflurane-anesthetized male Long-Evans rats (6-8 weeks old), and chilled in ice-cold ACSF (in mM: 1.25 NaH₂PO₄, 1.3 mM MgSO₄, 2.5 mM CaCl₂, 3.5 KCl, 15 glucose, 24 NaHCO₃, and 118 NaCl) bubbled with 95% O₂/5% CO₂ (pH=7.35). Acute transverse slices of dorsal hippocampus (400 μm thick) were recovered in an interface chamber at room temperature, as described previously. Tsokas, P. et al. (2005) 25 (2005). J Neurosci pp. 5833-5843. Slices were individually transferred to a recirculating submersion recording chamber and superfused with ACSF at 30-32° C. Field EPSPs (fEPSPs) were recorded with a ACSF-filled pipette (2-4 MS2) positioned in stratum radiatum of area CA1, and Schaffer collateral inputs were stimulated with 50 μs monophasic pulses using a bipolar concentric electrode placed in area CA3. Weak HFS, which normally induces only transient synaptic potentiation 19, consisted of two 1-s trains of 100 Hz pulses, delivered 20 s apart, with stimulus intensity set at 20% of the spike threshold.

IGF-II, freshly prepared from stock to a final concentration of 1 nM, was introduced 20 min before HFS was delivered and was present for the remainder of experiment. The antibody against the IGF-II receptor was used at a final concentration of 16 or 50 μg/mL, prepared fresh before use. As these two concentrations produced indistinguishable results, the data were pooled for analysis and presentation. The antibody was introduced at least 30 min before HFS was delivered, and remained in the superfusate for the rest of the experiment. The n's for the electrophysiology experiment are: Veh (wLTP: n=4; no stim.: n=4), IGF-II (wLTP: n=5; no stim.: n=6), IGF-II+anti-IGF-IIR antibody (wLTP: n=5; no stim.: n=5).

### Statistical Analysis.

One- or two-way analysis of variance (ANOVA) followed by either the Newman-Keuls, or Bonferroni post-hoc test, or Student's t-test was used for statistical analyses. For the electrophysiology experiments, EPSP slopes were compared by a Student's t-test.

### Example 1

### Measurement of IGF-II mRNA and Protein Expression Following Inhibitory Avoidance (IA) Training

IGF-II mRNA expression levels in the hippocampus were measured after IA training. Groups of rats underwent either

(1) IA training, described above, or
(2) the control behavioral protocols, which consisted of either
(a) exposure to the training apparatus without foot shock followed by euthanasia immediately after (0 h); or
(b) exposure to unpaired context and shock with euthanasia at a different time point (unpaired control).

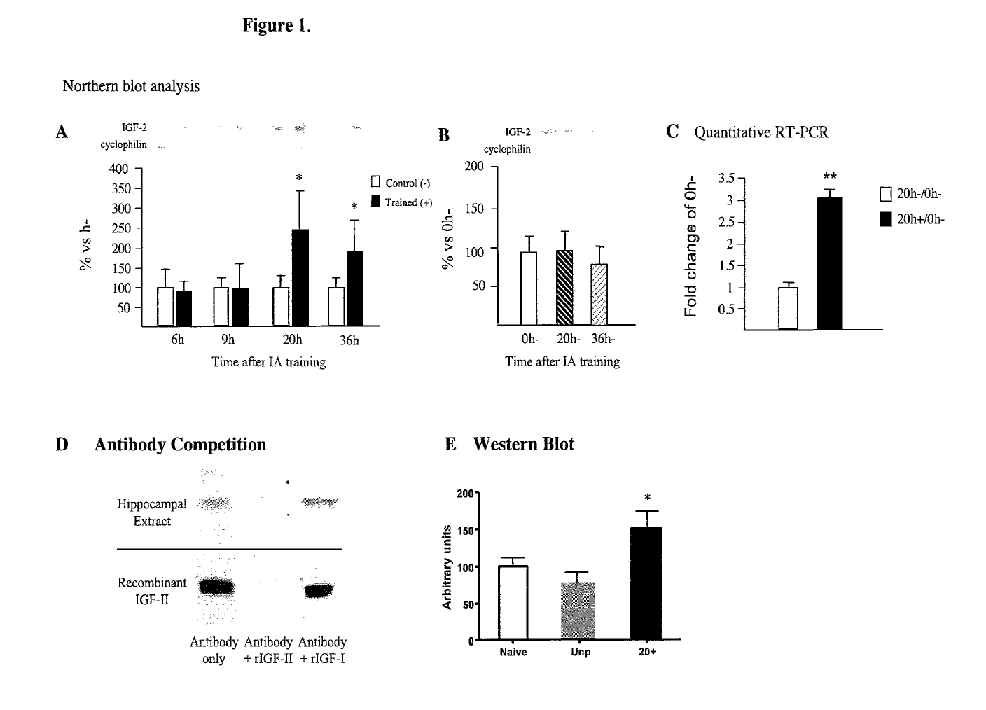

Both hippocampi of each animal were dissected out, pooled and treated as one sample, and all individual samples were extracted for mRNA analyses. As depicted in FIG. 1A, quantitative northern blot analysis revealed that, compared to their time-paired controls, there was no significant change at 6 and 9 hours (h) after training of IGF-II expression (6 h, 90.2±10.2%; 9 h, 98.6±59.5%). However, the IGF-II mRNA expression was significantly increased at 20 and 36 hours after training (20 h, 250.6±90.2%; 36 h, 194.7±61.0%, student t-test p<0.05).

Furthermore, in a separate experiment, it was found that neither the 20 h nor the 36 h control groups were significantly different from the 0 h control group (FIG. 1B, one-way ANOVA followed by Neuman-Keul post-hoc, p<0.01 n=6 per control group). Quantitative RT-PCR carried out on mRNA extracts (n=6 for all groups) obtained at 20 hours after either training or unpaired control protocol and compared to 0 h controls, confirmed the results found with northern blot analyses (FIG. 1C). In the same extracts, IGF-I mRNA remained unchanged.

In previous studies, it was shown that IA training induces a significant increase in hippocampal C/EBPβ, which starts between 6 and 9 h after training, lasts for at least 28 h and returns to baseline by 48 h after training. Experiments were performed to determine whether the IGF-II increase depends on C/EBPβ. Taubenfeld et al., (2001) Nat. Neurosci. 4, pp. 813-818, reported that an hippocampal bilateral injection of C/EBPβ antisense oligodeoxynucleotide (β-ODN), 5 h after IA training, blocks the training-dependent induction of C/EBPβ and completely disrupts memory consolidation. Experiments performed using this injection protocol and quantitative western blot analyses revealed that, compared to time-paired control injections of scrambled ODN (SC-ODN), 13-ODN completely disrupted the training-induced IGF-II increase without changing the IGF-II expression in unpaired control rats 24 h after training. Chromatin immunoprecipitation (ChIP) of hippocampal extracts confirmed that C/EBPβ binds in vivo to a C/EBPβ consensus sequence in the promoter region of the rat IGF-II exon 1. Thus, IA training leads to an increase in hippocampal C/EBPβ that regulates a downstream increase in the expression of IGF-II.

### Protein Expression

Western blot analyses were employed to determine whether the IGF-II mRNA changes were paralleled by changes in the IGF-II protein levels. Western blot experiments were performed with a commercially available anti-IGF-II antibody (Millipore, Billerica, Mass.). The antibody specifically recognizes IGF-II, as, the 14 kDa band recognized by this antibody in rat hippocampal extract, as well as recombinant IGF-II, were specifically competed by 25× excess amount of recombinant IGF-II, but not recombinant IGF-I (FIG. 1D).

Therefore, this anti-IGF-II antibody was used to perform quantitative western blot analyses of hippocampal extracts from rats that underwent training and were euthanized 20 hours later (FIG. 1E). Naïve and unpaired rats were used as controls. IGF-II levels were normalized against the relative concentration of actin. As shown in FIG. 1E, a one-way ANOVA followed by Newman Keul post-hoc test revealed a significant increase of IGF-II concentration in the hippocampi of trained rats (20+, 154.0±22.1%, n=12), compared to that of naïve (100.0±6.6%, n=12) and unpaired rats (unp, 89.2±13.6% n=8) (p<0.05). IGF-II protein levels were not increased at 72 h or 96 h after training, compared to naïve and unpaired animals.

### Example 2

### The Role of Hippocampal IGF-II In Memory Consolidation IGF-II Antisense Administration

IGF-II expression in the hippocampus was knocked down to determine if IGF-II has a role in IA memory consolidation. Hippocampal IGF-II expression was knocked-down by bilateral injections of antisense ODN (SEQ ID NO: 4) at various time points after IA training and the result of these treatments on IA long-term memory were determined

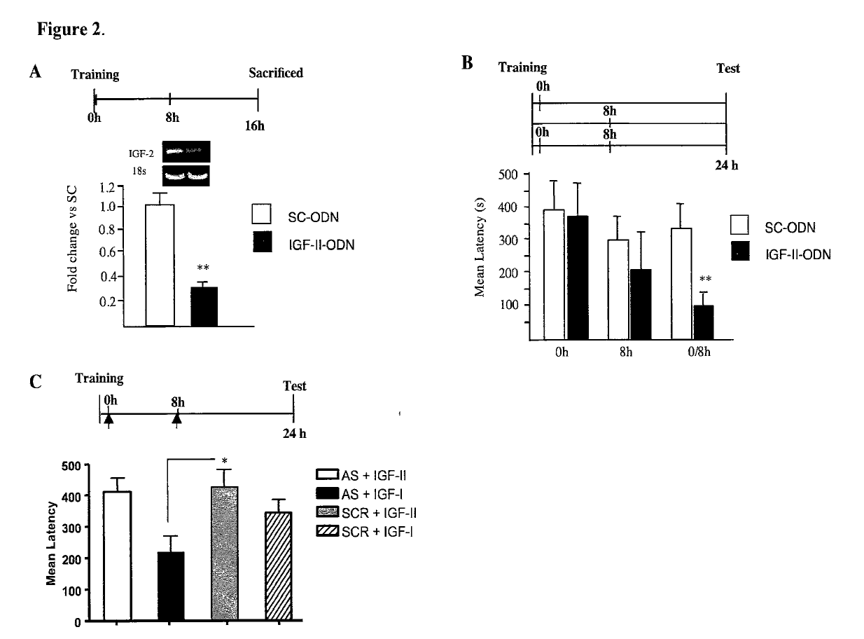

First, the effect of IGF-II antisense ODN (IGF-II-ODN, SEQ ID NO: 4) on the expression levels of IGF-II was investigated. Groups of rats were trained in IA and received a double injection bilaterally into the hippocampi of either IGF-II-ODN (SEQ ID NO: 4) (n=4) or a relative control SC-ODN (SEQ ID NO: 5) (n=4). The two injection time points were immediately after training and 8 hours later (FIG. 2A). Rats were euthanized 16 hours after training and both hippocampi per animal were dissected out, pooled and extracted for RNA analysis. Quantitative RT-PCR (qRT-PCR) was used to determine the expression levels of IGF-II. Compared to SC-ODN, IGF-II-ODN injections significantly decreased the levels of IGF-II mRNA, indicating that the antisense effectively knocked-down the mRNA concentration of IGF-II (SC-ODN: 1.0±0.1-fold; IGF-II-ODN: 0.3±0.04-fold, Student t test p<0.01 n=4 per group). These results indicate that double injections at immediately after IA training and 8 hours later significantly decrease the expression of IGF-II mRNA in the hippocampus.

Double injections delivered 8 hours apart were then used to test whether disrupting IGF-II expression in the hippocampus affects memory consolidation (FIG. 2B). Groups of rats (n=8) were trained in IA and injected both immediately after training and 8 hours later with either IGF-II-ODN or SC-ODN, and tested for memory consolidation 24 hours after training. The effect of each single injection was also measured (delivered either immediately after training or 8 hours later). A one-way ANOVA revealed a significant effect of treatment [F (5,43) p<0.001]. Newman Keul post-hoc analyses indicated that retention levels of rats that received a single bilateral injection of IGF-II-ODN either immediately after training or 8 hours later were not significantly different from those of respective SC-ODN injected controls (0 h: IGF-II-ODN: 381±103.42, 381; SC-ODN: 408.1±77.99; 8 h: IGF-II-ODN: 208.99±118.64; SC-ODN: 294.81±108.3). Although non-significant, the IGF-II-ODN-injected rats at 8 hours after IA training had a trend toward a memory deficit, compared to SC-ODN injected controls.

In contrast, a double injection of IGF-II-ODN at 0 h and 8 h after training resulted in a significant disruption of memory retention compared to a double injection of SC-ODN (IGF-II-ODN: 96.1±32.8 sec, SC-ODN 325.5±130.6 sec; p<0.001 n=8/group) (FIG. 2B). These results indicate that IGF-II plays a critical role after training for IA memory consolidation.

### Co-Administration of IGF-II Antisense and Recombinant IGF-II Protein

The ability of IGF-II to rescue the impairment of memory caused by injection of antisense IGF-II ODN was then tested, by the co-administration of IGF-II antisense and recombinant IGF-II protein. Groups of rats were injected immediately after training and 8 hours later with a mixture of either IGF-II-ODN or SC-ODN and recombinant IGF-II protein, or, as control, recombinant IGF-I (SEQ ID NO: 6). A one-way ANOVA revealed a significant effect of treatment [F(3,31)=3.49, p<0.05]. Newman-Keul post-hoc tests revealed that recombinant IGF-II, but not IGF-I, significantly restored the memory impairment caused by IGF-II-ODN treatment (FIG. 2C: IGF-II-ODN+rIGF-II: 410.03±46.02, IGF-II-ODN+rIGF-I: 224.28±52.16; p<0.05 n=8/group). These results indicate that IGF-II plays a role during IA memory consolidation.

### Example 3

### IGF-II Expression in the Hippocampus for Memory Consolidation—Time Experiment

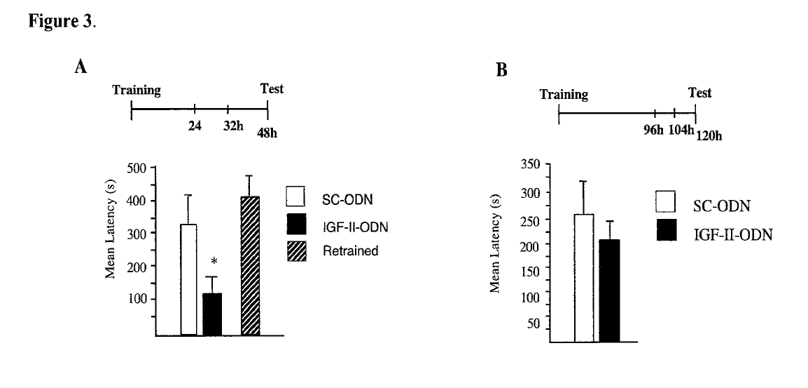

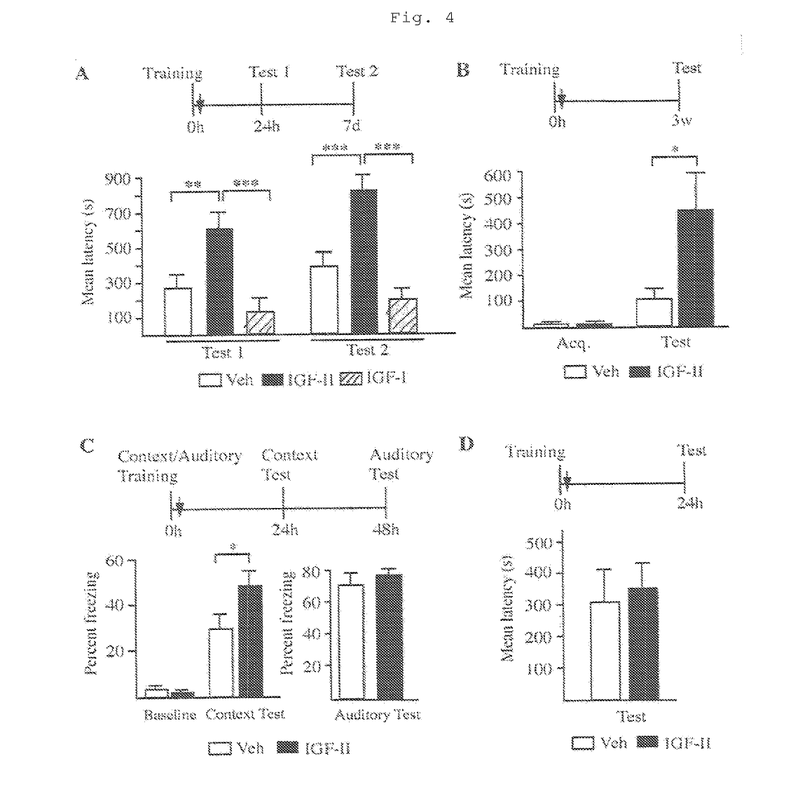

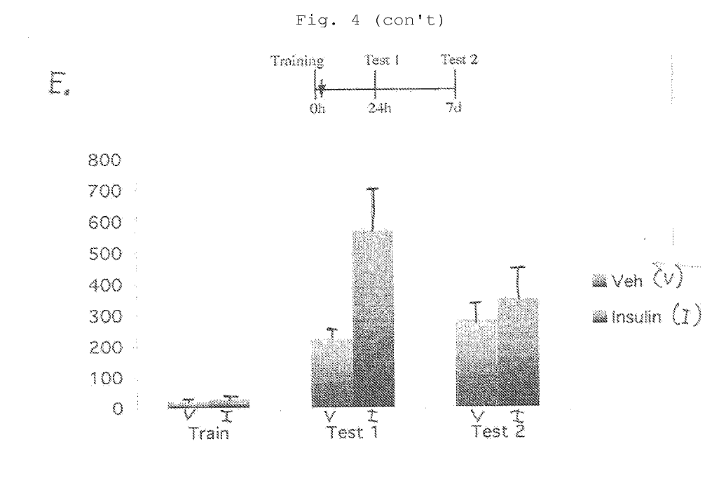

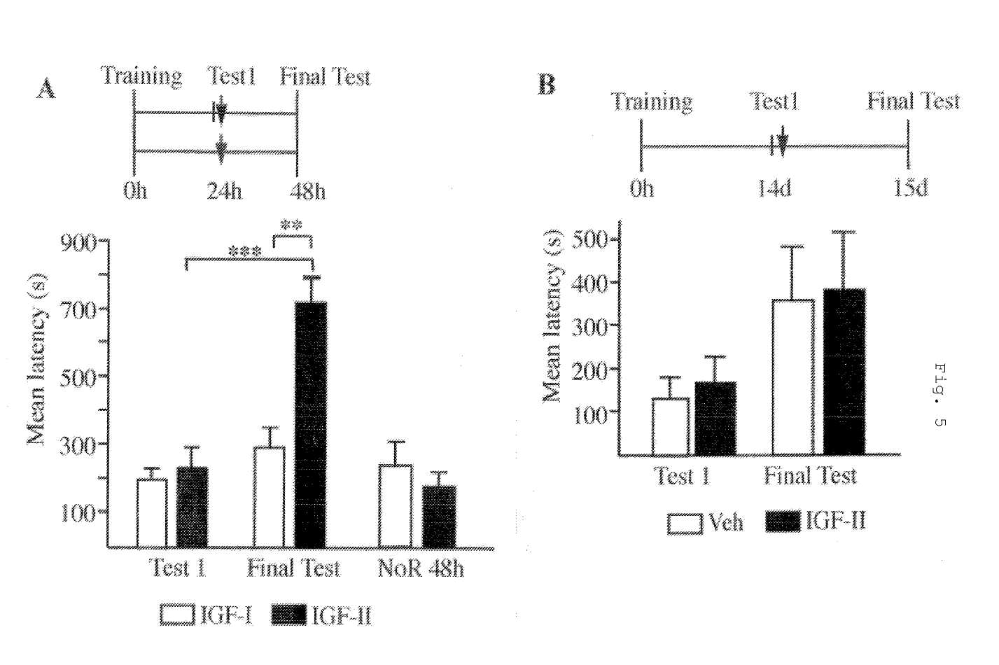

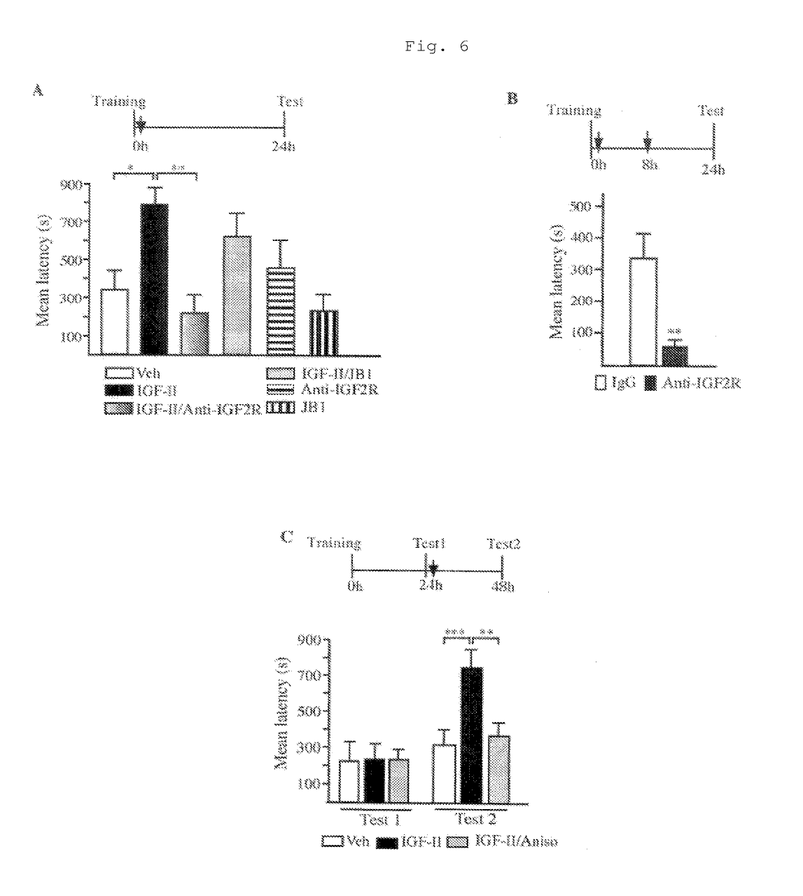

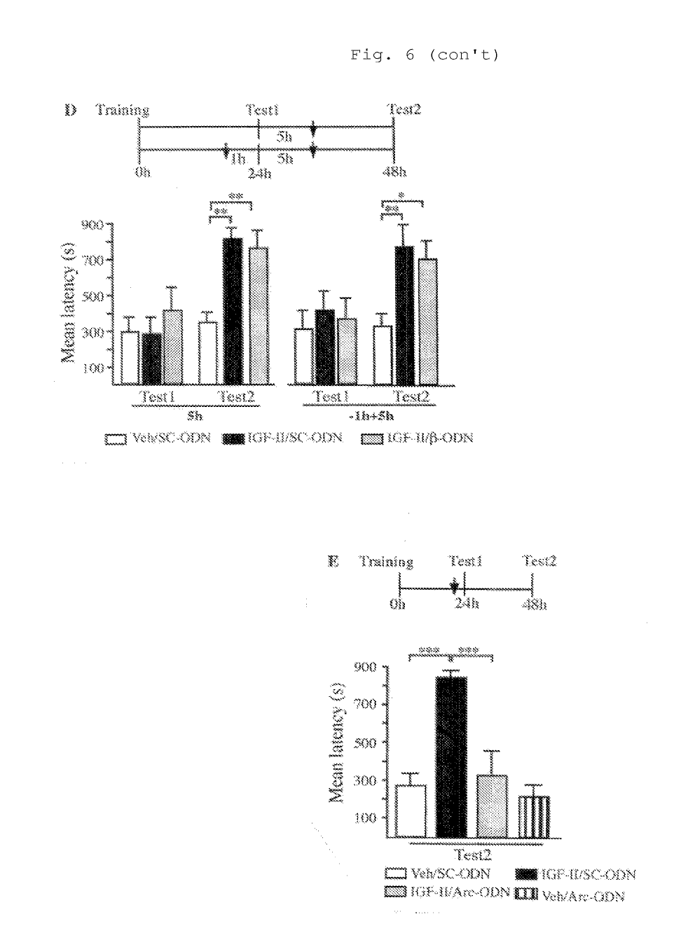

Bilateral double injections were delivered into the hippocampus of a group of rats (n8), 8 hours apart, starting at either 24 or 96 hours after training (FIG. 3). As depicted in FIG. 3A, compared to SC-ODN controls, injections of IGF-II-ODN delivered at 24 and 32 hours after training significantly impaired memory retention, when subjects were tested 48 hours after training (SC-ODN, 316.6±67.5 sec; IGF-II, 113.84±55.6 sec p<0.05, Student t test n=8/group).

To exclude that IGF-II-ODN-mediated memory disruption was due to a non-specific effect (e.g., hippocampal damage), the IG-II-ODN-injected, amnesic rats were re-trained in IA and tested 24 hours later. As shown in FIG. 3A (third bar), retrained animals had robust memory retention (Retrained: 413.15±53.21).

However, when rats received bilateral IGF-II-ODN double injections at 96 and 104 hours after training and were tested 120 hours after training (24 hours after the first injection), memory was intact (FIG. 3B: SC-ODN, 256.3±74.5 sec; IGF-II-ODB, 217.7±33.8 sec, n=6/group). Together, these results indicate that IGF-II plays an essential role during memory consolidation for more than one day after training. However, at later times, its function is no longer required.

### Example 4

### IGF-II Administration and its Effect on Memory Retention

The administration of IGF-II was tested to determine whether IGF-II modulates the strength of memory retention. To address this question, bilateral injections of either

(1) recombinant IGF-II (n=8),
(2) recombinant IGF-I (n=7), or
(3) equal volume of vehicle solution (Phosphate Buffered Saline) (n=7)
were delivered into the hippocampus of a group of rats, immediately after IA training. Latency cut-off time was raised to 900 sec.

Bilateral injections of IGF-II immediately after training significantly enhanced memory retention 24 h later, compared to IGF-I or vehicle solution (FIG. 4A). Memory enhancement was maintained at 7 days after training (FIG. 4A). Enhancement was not due to a non-specific locomotor effect, as IGF-II-treated rats showed similar locomotor activity compared to that of PBS or IGF-I treated animals. The IGF-II-mediated memory enhancement was dose-dependent as hippocampal injections of 25 or 2.5 ng, like 250 ng, given immediately after training significantly enhanced memory retention at 24 h but to different degrees. Moreover, bilateral hippocampal injection of IGF-II immediately after training resulted in a significant enhancement of memory retention 3 weeks after training when the latency of vehicle-injected rats was not significantly different from acquisition, indicating that IGF-II prevents forgetting (FIG. 4B)

The IGF-II effect generalized to another memory tasks, contextual fear conditioning. Bilateral hippocampal injection of IGF-II immediately after contextual-auditory fear conditioning training significantly enhanced contextual fear conditioning retention tested 24 h later, without affecting auditory fear conditioning tested 48 h after training (FIG. 4C). No difference in baseline freezing was observed between groups prior to footshock delivery (FIG. 4C).

Because IA consolidation also involves the amygdala, experiments were conducted to test the effect of bilaterally injecting IGF-II into the amygdala immediately after training. No significant effect was found at testing, 24 h after training (FIG. 4D).

Animals administered bilateral hippocampal injections of insulin immediately after IA training in the same protocol set out above exhibited enhanced memory strength at 24 h but enhancement was not maintained at day 7 (FIG. 4E).

These results demonstrate IGF-II acts in the hippocampus to promote memory enhancement and prevent forgetting.

### Example 5

### IGF-II Significantly Enhances Memory Strength if Administered After Retrieval

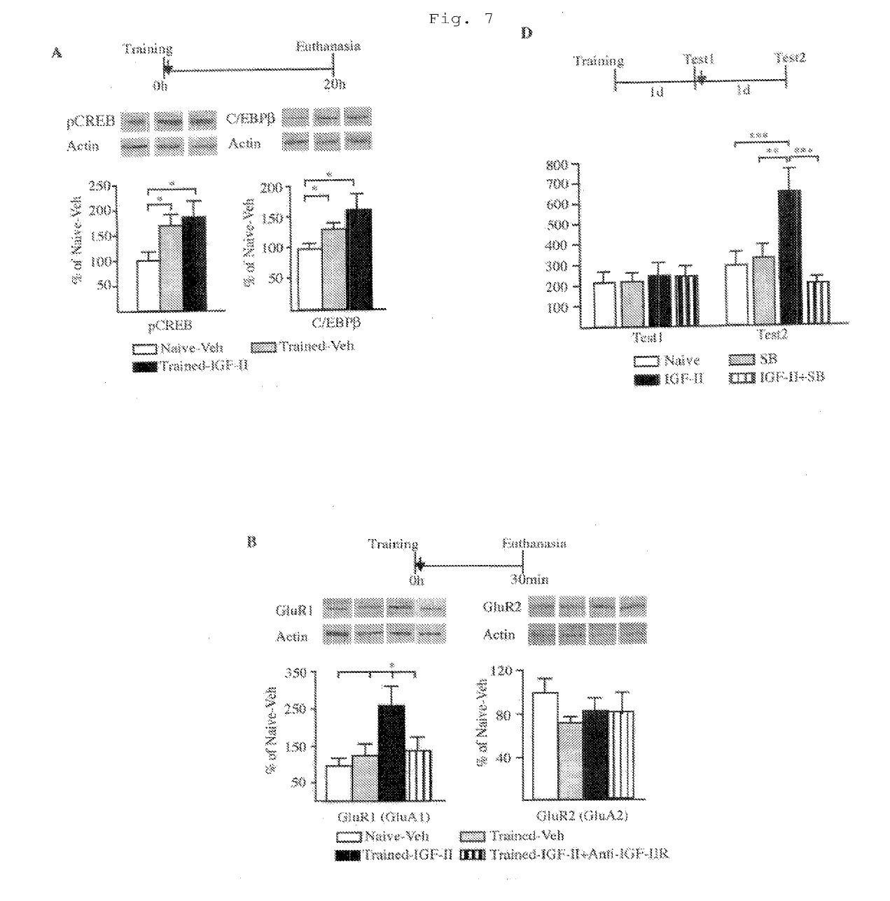

An established memory, insensitive to several types of interferences, becomes labile and undergoes another protein-synthesis-dependent reconsolidation process if it is reactivated, for example, by a non-reinforced retrieval event. Bilateral hippocampal injection of IGF-II 24 h after training had no effect on memory retention tested 48 h after training (FIG. 5A). However, if 24 h after training memory was reactivated by testing (Test1), similar IGF-II injections significantly enhanced memory retention tested 24 h later (Final Test, FIG. 5A).

Studies in IA and certain other types of learning have shown that reconsolidation is temporally limited. IA memory undergoes protein-synthesis-dependent reconsolidation if retrieval occurs at 2 or 7 days after training but not at 2 or 4 weeks after training. We determined whether the enhancing effect of IGF-II was also temporally limited, and coincided with the reconsolidation-sensitive temporal window. Bilateral hippocampal injection of IGF-II immediately after a reactivation by testing (Test1) that occurred two weeks after training had no effect, compared to vehicle, on memory retention tested one day later (Final Test) (FIG. 5B). Hence, hippocampal IGF-II-mediated memory enhancement occurred only within a sensitive temporal window, which overlaps with the limited period during which IA memory undergoes reconsolidation.

### Example 6

### Memory Enhancement Requires IGF-II Receptors, De Novo Protein Synthesis and Activity-Regulated Cytoskeletal Protein (Arc) but not IGF-I Receptors or C/EBPβ

IGF-II activates both IGF-I and IGF-II receptors but with different affinity. To determine whether IGF-II-mediated memory enhancement recruits one or both of these receptors, we tested the effect of IGF-I and IGF-II receptor (R) selective inhibitors. Specific inhibitors of either IGF-IR (JB-1) or IGF-IIR (anti-IGF-IIR antibody) injected together with IGF-II into the hippocampus completely abolished the memory enhancement compared to respective controls (FIG. 6A). The inhibitors administered without IGF-II did not affect memory retention (FIG. 6A). Hence, IGF-II—but not IGF-I receptors mediated the IGF-II-evoked memory enhancement.

Similarly to the antisense experiments, the single injection of anti-IGF-IIR antibody had no effect on memory retention; thus, we tested whether a sustained blockade of IGF-II receptor is required to affect memory consolidation. Bilateral hippocampal double injections of the anti-IGF-IIR antibody immediately after training and 8 hours later completely disrupted memory retention at 24 h after training, compared to IgG-injected controls (FIG. 6B).

We next asked whether IGF-II-mediated memory enhancement recruits new protein synthesis. Since memory consolidation per se requires new protein synthesis in the hippocampus, blocking protein synthesis in IGF-II-injected rats after training would not be informative. However, since it has been shown that new protein synthesis is not required in the hippocampus for IA reconsolidation, we could test the effect of protein synthesis inhibition on retrieval-dependent IGF-II-mediated enhancement.

Bilateral hippocampal co-injection of IGF-II and the protein synthesis inhibitor anisomycin immediately after Test 1, 24 h after training, revealed that anisomycin compared to vehicle completely disrupted the IGF-II-mediated memory enhancement tested 24 later (FIG. 6C) without changing the training-induced retention levels. Hence, memory enhancement, but not reconsolidation, required hippocampal de novo protein synthesis.

To begin identifying which proteins participate in the memory enhancement, we investigated the role of C/EBPβ. Bilateral hippocampal injection of β-ODN 5 h after memory reactivation (Test1) had no effect on the IGF-II-mediated memory enhancement tested 48 h after training (FIG. 6D). The timing of the ODN injections was based on previous kinetics studies showing maximal disruptive effect of β-ODN at this time point in the hippocampus on memory consolidation and in the amygdala on reconsolidation. To test whether a prolonged antisense treatment affected post-retrieval IGF-II-mediated memory enhancement, bilateral double injections into the hippocampus of either β-ODN or SC-ODN, were delivered at 1 h before and 5 h after reactivation (Test1). This treatment also completely failed to affect IGF-II-evoked memory enhancement (FIG. 6D), suggesting that, although de novo protein synthesis is critical for memory enhancement, C/EBPβ is not.

Protein synthesis-mediated enhancement may recruit synaptic rather than cell-wide, transcriptional mechanisms. One rapidly regulated translation known to occur at activated synapses and be essential for long-term plasticity and memory is that of activity-regulated cytoskeletal-associated protein (Arc). Bilateral hippocampal injection of Arc antisense (Arc-ODN), compared to relative SC-ODN, 1 h before reactivation (Test1), completely blocked the post-retrieval IGF-II-mediated memory enhancement tested 2 d after training (FIG. 6E). However, the same treatment did not affect training-induced memory retention (FIG. 6E).

Together, these results demonstrate that IGF-II-mediated enhancement requires IGF-II but not IGF-I receptors. Furthermore, retrieval-dependent IGF-II-mediated enhancement required de novo protein synthesis and Arc but not C/EBPβ, suggesting that it may use synaptic rather than cell-wide-regulatory mechanisms.

### Example 7

### IGF-II-Dependent Memory Enhancement Correlates with the Activation of Glycogen Synthase Kinase 3 (GSK-3), a Significant Increase in Synaptic GluR1 Subunits of α-amino-3-hydroxy-5-methyl-4-isoxazolepropionic Acid (AMPA) Receptors but not with the Activation of the CREB-C/EBP Pathway

During memory consolidation the CREB-C/EBP-dependent cascade is significantly activated. In IA, both CREB phosphorylation in ser 133 (pCREB) and the expression of C/EBPβ are significantly increased for more than 20 hours after training We examined whether IGF-II mediated memory enhancement following training correlates with an enhanced hippocampal activation of the CREB-C/EBP pathway. Quantitative western blot analyses of hippocampal extracts at 20 h after training revealed that, compared to naïve rats, training significantly increased both pCREB and C/EBPβ (trained-vehicle vs. naïve-vehicle, FIG. 7A). Compared to vehicle, IGF-II treatment immediately after training resulted in only a tendency toward a further increase, which was not statistically significant (FIG. 7A). Thus, IGF-II-mediated memory enhancement did not correlate with significant enhancement in the activation of the CREB-C/EBP cascade, indicating that IGF-II-regulated mechanisms may be synaptic rather than cell-wide. We therefore investigated the synaptic expression levels of GluR1 and GluR2 AMPA receptor subunits. Synaptic GluR1 AMPA receptors have been shown to be rapidly increased following IA training and play a role in its consolidation. Furthermore, AMPA receptor subunit trafficking is known to accompany long-term plasticity in LTP and long-term depression (LTD).

As shown in FIG. 7B, quantitative western blot analyses of synaptoneurosomal extract obtained 30 minutes after training revealed that, compared to naïve, trained rats had an increased expression of synaptic GluR1, which, however, was not significant, probably due to the relatively low shock intensity used in our experiments. The levels of GluR1 were significantly increased in trained rats treated with IGF-II compared to vehicle. This increase was completely abolished by anti-IGF-IIR antibody. GluR2 levels remained unchanged across groups.

Previous studies reported that AMPA receptor trafficking and dendritic expression of GluR1 in neurons are regulated by GSK-3 and, interestingly, IGF-II has been implicated in GSK-3 regulation. As depicted in FIG. 7C, the IGF-II-mediated significant increase of GluR1 was paralleled by a significant synaptic activation of GSK-3β (measured by its dephosphorylation at ser 9), which was also completely abolished by anti-IGF-IIR antibody. Furthermore, blocking GSK-3 function with pretraining hippocampal injection of the inhibitor SB216763 completely blocked IA memory consolidation (data not shown), whereas hippocampal injection of SB216763 immediately after memory reactivation (Test1) completely and selectively eliminated the IGF-II-mediated enhancement tested 2 d after reactivation (Test2) without affecting reconsolidation (FIG. 7D).

Thus, IGF-II-dependent memory enhancement required the activation of GSK-3β and correlated with an increased synaptic expression of GluR1.

### Example 7

### IGF-II Enables Long-Term Potentiation (LTP)

To determine whether the effect of IGF-II was generalized to long-term synaptic plasticity, we tested the effect of IGF-II on hippocampal LTP, which is widely regarded as a cellular correlate of long-term memory. IGF-II was applied to acute hippocampal slices and both LTP in the Schaffer collateral pathway and basal synaptic transmission were investigated. As shown in FIG. 7E, a weak tetanus elicited a transient synaptic potentiation that decayed to baseline within 100 minutes after induction. When this weak stimulus was delivered in the presence of IGF-II, stable LTP was expressed. This enabling effect was completely blocked in slices that were pretreated with anti-IGF-IIR antibody (FIG. 7E). Neither IGF-II nor the anti-IGF-IIR antibody affected basal synaptic transmission.

### Discussion

Identifying memory enhancers and understanding their mechanisms is a most important goal for the learning and memory field and the development of cognitive enhancement therapies. The results above demonstrated that memory retention can be enhanced, LTP promoted and forgetting prevented by the administration of IGF-II, a growth factor physiologically regulated following learning, downstream of C/EBPβ, and required for memory consolidation. IGF-II was effective when administered during a temporally-restricted phase induced by either learning or memory retrieval. The IGF-II-dependent effect was mediated by IGF-II receptors, required de novo protein synthesis, the activation of GSK3 and the expression of Arc and was paralleled by an increase of synaptic GSK3β activation and GluR1 expression.

### The Regulation and Functional Requirement of IGF-II During the Initial Phase of Consolidation

The results reported in the Examples show that the expression of IGF-II was upregulated in the hippocampus at 20 and 36 h but not at 72 and 96 h after IA training. This upregulation required C/EBPβ, which plays an essential, evolutionarily conserved role in memory consolidation within the CREB-dependent gene cascade. In line with its post-training temporal expression regulation, IGF-II was required for memory consolidation during the first 1-2 days after training but not later, as its antisense-mediated knock-down results in amnesia. The antisense-mediated memory disruption was not due to toxicity or nonspecific effects, since the treatment only affected memory retention during a specific time window and had no effect at other time points. Moreover, rats with antisense-mediated memory impairment had normal memory retention after retraining, indicating that their hippocampi were fully functional.

Training-induced IGF-II increase required C/EBPβ; therefore, learning-dependent hippocampal IGF-II regulation is a downstream, or target event of the recruitment of C/EBPβ. C/EBPβ appears to directly regulate the transcription of IGF-II because hippocampal C/EBPβ binds to a consensus sequence present in the promoter region of the rat IGF-II exon 1 and previous data reported that, in many species, IGF-II promoters bear several putative C/EBP-binding sites. In agreement with the temporal window of expression regulation, IGF-II, like C/EBPβ, plays a critical role in memory consolidation between 9 and 24-48 h after training, but not later. The results reported herein confirm and extend the conclusion that the transcription- and translation-dependent phase of IA consolidation in the dorsal hippocampus lasts for more than one day, but less than 2-4 days.

### IGF-II is a Potent Memory Enhancer

An acute hippocampal administration of IGF-II significantly and persistently enhanced memory retention if given in concert with either training or retrieval. This effect was paralleled by a significant and persistent LTP stabilization. The same treatment also prevented forgetting. Remarkably, the effect of IGF-II was significant under an acute treatment and with low doses, which is uncommon in memory enhancement experiments thus far reported, and is important for clinical applications. The effect was generalized to different types of hippocampal-dependent memories.

The enhancing effect of IGF-II was temporally limited following training: injections of IGF-II 24 h after training did not change memory retention. Antisense-mediated disruption of IGF-II at 24 and 32 h, however, after training impaired memory retention. Hence, although endogenous IGF-II synthesis was still required for memory consolidation, its exogenous administration 24 h after learning was no longer effective in promoting memory enhancement. The memory-enhancing effect of IGF-II was thus restricted to an active phase that begins with training and lasted for less than 24 h. The enhancing effect of IGF-II re-emerged, however, at 24 h after training if memory was reactivated by retrieval, which is known to induce a gene-expression-dependent reconsolidation process. The IGF-II effect on retrieval was also temporally limited, as it is restricted to a temporal window that overlapped with the reconsolidation of IA, which lasted more than one but less than two weeks after training and does not take place in the hippocampus but in the amygdala7. The present studies show that the same temporal boundary applied to the IGF-II-dependent memory enhancement that targets the hippocampus.

Hippocampal new protein synthesis was not required for memory reconsolidation, but was necessary for the IGF-II mediated memory enhancement. This new protein synthesis included the expression of the immediate early gene Arc, which is known to critically mediate long-term plasticity and memory consolidation.

### Molecular Mechanisms of IGF-II-Mediated Memory Enhancement

The effect of IGF-II was selective and specific for the fragment Ala25-Glu91 of IGF-II and does not occur with an analogous fragment of IGF-I. Moreover, it was selectively mediated by IGF-II and not IGF-I receptors. In agreement, IGF-IIRs were required for memory consolidation.

The effect of IGF-II in mediating memory enhancement was not paralleled by significant activation of pCREB and C/EBPβ and did not functionally require C/EBPβ expression, but depended on GSK-3 and Arc and was accompanied by a significant increase in synaptic GSK3β activation and GluR1 expression. Since C/EBPβ is significantly upregulated for more than 28 h after training, it is possible that the already synthesized C/EBPβ is sufficient to mediate memory enhancement. Alternatively, the enhancement may recruit mechanisms either downstream of C/EBPβ or distinct from those mediating consolidation. The results with Arc, GSK-3, and GluR1 suggest that IGF-II targets synaptic mechanisms, possibly those in activated synapses. These results are in line with previous studies showing a functional link between GSK-3 activity and dendritic clustering of GluR134, as well as that of Arc expression and the membrane trafficking of GluR1, synaptic plasticity and memory consolidation.

IGF-II readily crosses the blood-brain barrier. Reinhardt et al., (1994). Endocrinology 135 pp. 1753-1761. Based on the results reported herein, IGF-II is a novel target for cognitive enhancement therapies.

Patents, patent applications, publications, product descriptions, and protocols which are cited throughout this application are incorporated herein by reference in their entireties.

The embodiments illustrated and discussed in this specification are intended only to teach those skilled in the art the best way known to the inventors to make and use the invention. Nothing in this specification should be considered as limiting the scope of the present invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A method of enhancing memory strength in a subject in need thereof, comprising administering to the subject, a therapeutically effective amount of a compound selected from insulin-like growth factor II protein (IGF-II), a nucleic acid encoding IGF-II protein, an IGF-II peptide, a nucleic acid encoding an IGF-II peptide, or a combination thereof.

2. The method of claim 1, wherein the memory is a long-term memory.

3. The method of claim 1, wherein the compound is administered by a route selected from the group consisting of intranasal, oral, topical, anal, ocular, optic, intramuscular, intraperitoneal, intravenous, intracerebral, and any combination thereof.

4. The method of claim 1, wherein the IGF-II protein comprises the sequence identified as SEQ ID NO: 1.

5. The method of claim 1, wherein the IGF-II protein is recombinantly produced.

6. The method of claim 1, wherein the IGF-II protein or the IGF-II encoding nucleic acid is formulated in a pharmaceutical composition which comprises a pharmaceutically acceptable carrier or excipient.

7. The method of claim 1, wherein the subject does not have a memory impairment.

8. The method of claim 1, wherein the subject has a memory impairment.

9. The method of claim 8, wherein the memory impairment is associated with a neurodegenerative disease or aging.

10. The method of claim 9, wherein the neurodegenerative disease is selected from the group consisting of Huntington's disease, Parkinson's disease, amyotrophic lateral sclerosis (ALS), neurodegeneration due to aging, and Alzheimer's disease.

11. The method of claim 8, wherein the memory impairment is associated with head injury, spinal cord injury, seizure, stroke, epilepsy, ischemia, neuropsychiatric syndromes, CNS damage resulting from viral encephalitis, CNS damage resulting from meningitis, or CNS damage resulting from a tumor.

12. The method of claim 1, further comprising memory reactivation.

13. The method of claim 11, wherein the IGF-II compound is administered concomitant with the memory reactivation.

14. A method for treating memory impairment in a subject in need thereof, comprising administering to the subject, a therapeutically effective amount of a compound selected from insulin-like growth factor II protein (IGF-II), a nucleic acid encoding IGF-II protein, an IGF-II peptide, a nucleic acid encoding an IGF-II peptide, or a combination thereof.

15. The method of claim 14, wherein the memory is a long-term memory.

16. The method of claim 14, wherein the compound is administered by a route selected from the group consisting of intranasal, oral, topical, anal, ocular, optic, intramuscular, intraperitoneal, intravenous, intracerebral, and any combination thereof.

17. The method of claim 14, wherein the IGF-II protein comprises the sequence identified as SEQ ID NO: 1.

18. The method of claim 14, wherein the IGF-II protein is recombinantly produced.

19. The method of claim 14, wherein the IGF-II protein or IGF-II encoding nucleic acid is formulated in a pharmaceutical composition which comprises a pharmaceutically acceptable carrier or excipient.

20. The method of claim 14, wherein the memory impairment is associated with a neurodegenerative disease or aging.

21. The method of claim 20, wherein the neurodegenerative disease is selected from the group consisting of Huntington's disease, Parkinson's disease, amyotrophic lateral sclerosis (ALS), neurodegeneration due to aging, and Alzheimer's disease.

22. The method of claim 14, wherein the memory impairment is associated with head injury, spinal cord injury, seizure, stroke, epilepsy, ischemia, neuropsychiatric syndromes, CNS damage resulting from viral encephalitis, CNS damage resulting from meningitis, or CNS damage resulting from a tumor.

23. A method of treating or preventing memory decay in a subject with mild cognitive impairment, comprising administering to the subject, a therapeutically effective amount of a compound selected from insulin-like growth factor II protein (IGF-II), a nucleic acid encoding IGF-II protein, an IGF-II peptide, a nucleic acid encoding an IGF-II peptide, or combination thereof.

24. The method of claim 23, wherein the memory is a long-term memory.

25. The method of claim 23, wherein the composition is administered by a route selected from the group consisting of intranasal, oral, topical, anal, ocular, optic, intramuscular, intraperitoneal, intravenous, intracerebral, and any combination thereof.

26. The method of claim 23, wherein the IGF-II protein comprises the sequence identified as SEQ ID NO: 1.

27. The method of claim 23, wherein the IGF-II protein is recombinantly produced.

28. The method of claim 23, wherein the IGF-II protein or IGF-II encoding nucleic acid is formulated in a pharmaceutical composition which comprises a pharmaceutically acceptable carrier or excipient.

29. A method for disrupting memory in an experimental animal, comprising administering to the animal a composition comprising an IGF-II antisense oligonucleotide.

30. The method of claim 29, wherein the IGF-II antisense oligonucleotide comprises the nucleic acid sequence identified as SEQ ID NO: 4.

31. The method of claim 29, wherein the experimental animal is used to model Alzheimer's disease.

32. The method of claim 29, wherein the compound is administered by a route selected from the group consisting of intranasal, oral, topical, anal, ocular, optic, intramuscular, intraperitoneal, intravenous, intracerebral, and any combination thereof.

33. The method of claim 32, wherein the composition is administered by one or more intracerebral injections.

34. The method of claim 32, wherein the composition is administered by the intranasal route.
